(19) United States
(12) Patent Application Publication
(10) Pub. No.: US 2017/0232035 A1
(43) Pub. Date: Aug. 17, 2017

(54) Polygalacturonan Rhamnogalacturonan1 (PGRG1) Composition

(71) Applicant: Pharmagenesis, Inc., Redwood City, CA (US)

(72) Inventors: John H MUSSER, San Carlos, CA (US); Jinhua AN, Santa Clara, CA (US)

(73) Assignee: Pharmagenesis, Inc., Redwood City, CA (US)

(21) Appl. No.: 15/502,971

(22) PCT Filed: Aug. 17, 2015
(86) PCT No.: PCT/US2015/045574

§ 371 Date: Feb. 9, 2017

Related U.S. Application Data
(60) Provisional application No. 62/038,583, filed on Aug. 18, 2014.

(51) Int. Cl. A61K 31/732; A23L 33/105; A61K 45/06; A61K 36/481; C08B 37/00

(52) U.S. Cl. CPC A61K 31/732; A61K 36/481; C08B 37/0048; A61K 45/06; A23L 33/105; A61K 2236/33; A61K 2236/13; A61K 2236/15; A61K 2236/17; A23V 2002/00

(57) ABSTRACT

The present disclosure relates to polygalacturonan rhamnogalacturonan (PGRG1) compositions, as well as methods of making and methods of using said compositions in medicinally useful and pharmaceutically useful forms. Specifically, the present disclosure provides purified PGRG1 compositions isolated from roots of the Astragalus genus of plants, and more particularly from the species Astragalus membranaceus, as well as PGRG1 compositions having a weight average molecular weight of at least 40 kiloDaltons (kDa).

### TECHNICAL FIELD

The present disclosure relates generally to botanical compositions and methods for transforming natural botanical materials into medicinally useful and pharmaceutically acceptable compositions comprising polygalacturonan rhamnogalacturonan (PGRG1), as well as to methods of making and methods of using said compositions. Specifically, the present disclosure provides purified PGRG1 compositions isolated from roots of the Astragalus genus of plants, and more particularly from the species Astragalus membranaceus, as well as PGRG1 compositions having a weight average molecular weight of at least 40 kiloDaltons (kDa).

### BACKGROUND

Astragalus is a large genus (family Leguminosae) of annual and perennial flowering herbs and small shrubs found widely distributed all over the world. Astragalus plants have been used for centuries in traditional Chinese and Persian medicine, gaining popularity in the United States in the 1980s. In China, Astragalus is one of 50 fundamental herbs used in systems of traditional Chinese medicine, and is known as huáng qi, [“Wang Qi,” “huang-qi” or “huangqi” ( [symbol] ); b\check{e}i qi ( [symbol] ) and huáng qi huã huáng qi ( [symbol] )]. Astragalus is often used in combination with other herbs, such as ginseng, angelica, and licorice, to support and enhance the immune system, and it is still widely used in China for chronic hepatitis and as an adjunctive therapy for cancer. It is also used as a folk or traditional remedy for colds and upper respiratory infections, and for heart disease.

Common names for A. membranaceus (syn. A. propinquus) include milkvetch, locoweed and goat's-thorn (A. gummifer, A. tragacanthus). Preparations of the dried root of Astragalus mongholicus (“Huangqi”) are the most commonly used Astragalus species in Chinese medicine.

While there are over 2,000 species of Astragalus, the two species most commonly used medicinally are Astragalus membranaceus and Astragalus mongholicus. For example, A. membranaceus has been used to treat pulmonary and gastric diseases, as well as an antiperspirant and a diuretic. The roots of Astragalus membranaceus are used as a tonic asserted to increase metabolism and sweating, promote healing, and reduce fatigue, improve the functioning of the lungs, adrenal glands and the gastrointestinal tract. Astragalus membranaceus also is an active component in the herbal and mineral complex registered in Croatia as a food supplement Lectranal® used in treatment of seasonal allergic rhinitis. Radix Astragali is the whole dried root of Astragalus mongholicus Bunge var. Dahuricus (DC.) Podlech and var. Mongholicus. Constituents of Radix Astragali include polysaccharides, triterpenoids (astragalosides) as well as isoflavones (including kumatakenin, calycosin and formononetin) along with their glycosides and malonates. The polysaccharide compositions of Radix Astragali extract have been analyzed and characterized (Xu, et al., 2008, “Molecular weight and monosaccharide composition of Astragalus polysaccharides.”Molecules. 13(10):2408-2415).

Preparations of Astragalus have also been used in cancer therapy; for example, tragacanth, the gummy sap of milk vetch root, and swainsonine, a water-soluble indole alkaloid produced by several plants known as locoweeds, including milk vetch, have been used pharmaceutically. The natural gum tragacanth, used in pharmaceuticals and textiles, is made from several species of Astragalus occurring in the Middle East, including A. adscendens, A. gummifer, A. brachycalyx, and A. tragacanthus. Other Astragalus species are being investigated for their anti-cancer properties, such as the Turkish species, which include Astragalus brachypterus, Astragalus cephalotes, Astragalus microcephalus and Astragalus trojanus. Astragalus tongolensis and Astragalus scaberrimus have been used to remedy diarrhea, muscular numbness, poor circulation, weak lungs, asthma, nervousness, syphilis, scrofula and gall trouble. The seeds of Astragalus multiceps and Astragalus harmosus are used for colic, leprosy, emollient and demulcent.

Biotechnology firms also have been attempting to identify active components in Astragalus. Extracts of Astragalus propinquus (syn. A. membranaceus) are marketed as life-prolonging extracts for human use; for example, a proprietary extract of the dried root called TA-65 containing the saponin “cycloastragenol” (a.k.a. “TAT2”), “was associated with a significant age-reversal effect in the immune system, in that it led to declines in the percentage of senescent cytotoxic T cells and natural killer cells after six to twelve months of use” (Harley, et al., 2011, Rejuvenation Research 14 (1): 45-56). Furthermore, cycloastragenol is being studied to help combat HIV, as well as infections associated with chronic diseases or aging. Research at the UCLA AIDS Institute focused on the function of cycloastragenol in the aging process of immune cells, and its effects on the cells' response to viral infections. Cycloastragenol appears to increase the production of telomerase, an enzyme that mediates the replacement of short bits of DNA known as telomeres, which protect the ends of chromosomes and play a key role in cell replication, including in cancer processes (Fauce, et al., 2008, J. Immunology 181(10):7400-7406).

Although several parts of the plant seem to contain active constituents, it is most often the root that is used in herbal formulas. Astragalus plants are a rich source of polysaccharides, saponins, isoflavonoids and flavonoids. The polysaccharides originating from Astragalus are called “astragalans.” The saponins (or triterpene glycosides, also called “astragalosides”), isoflavonoids, and flavonoids consist of aglycons or glycosides. The amounts of astragalosides, trigonosides, and flavonoid constituents of dried root can vary according to the age, size and growing conditions of the root. Astragalus species are also being investigated for their usefulness in developing adjuvants for vaccines, as plant-derived saponins have been reported to stimulate secretion of a broad range of cytokines, possibly by triggering innate immunity. (Song and Hu, 2009, “Adjuvant activities of saponins from traditional Chinese medicinal herbs.” Vaccine. 27(36):4883-90).

Ubiquitous in plants is the cell wall component pectin. Pectins are a highly heterogeneous class of GalA-containing polysaccharides that are abundant in the plant cell wall. Pectic polysaccharides include the structural classes homogalacturonan (HG), xylogalacturonan (XGA), apiogalacturonan (AGA), and branched chain polysaccharides called rhamnogalacturonans (RGs). Of the pectic polysaccharides, the most abundant is homogalacturonan (HG), a homopolymer of (1-4)-α-D-galacturonic acid (GalA) residues that may be methylesterified and acetylated; HG comprises around 70% of pectin, with RG-I comprising approximately 35% and RG-II about 10% of pectin.

RGs are a group of closely related cell wall pectic polysaccharides; the two kinds of RGs are rhamnogalacturonan type I (also known as “RG-I” or “RGI”) and rhamnogalacturonan type II (“RG-IP” or RGII”) (Zakharova, et al., (2013) Organic Letters 15(8):1826-1829). The chemical structure of RG-I is complex, having a backbone consisting of repeating and alternating α-linked L-rhamnose and D-galacturonic acid disaccharide units with numerous branches of arabinans, galactans, or arabinogalactans positioned at C-4 of the rhamnose residues (McNeil, et al., 1980, Plant Physiol., 66:1128-1134; Zakharova, et al., 2013, Organic Letters 15(8):1826-1829). Thus, the backbone structure of rhamnogalacturonan I (RG-I) contains the repeating disaccharide unit: [→α-D-GalpA-1,2-α-L-Rhap-1,4→]ₙ (Caffall, et al., 2009, Carb. Res. 344:1879-1900).

The structural complexity of pectins imparts diverse physical and biochemical properties associated with certain interesting biological and industrial functions. Significant effort has been devoted to manipulating the quality and quantity of pectin and other wall polysaccharides through genetic manipulation and conventional breeding techniques as well as chemical synthesis (Bacic, 2006, Proc. Natl. Acad. Sci. 102(15):5639-5640).

However, the complex chemical nature of many botanical compositions can make them difficult to use in a controlled and predictable manner. Herbal medicines, produced from botanical materials, have presented a unique problem for manufacturers desiring the control, reproducibility, and standardization that are required of pharmaceuticals. The plurality of components contained in an herbal medicine and the large variation in composition and potency due to raw material growing and harvesting conditions can lead to unpredicted variations in chemical composition of different batches of botanical preparations obtained, making such preparations unsuitable for use in clinical situations.

Thus, despite the long history of use of preparations of Astragalus species as medicines, a need remains for the claimed, medicinally useful compositions as described herein.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

In one aspect, an isolated polygalacturonan rhamnogalacturonan 1 (PGRG1) composition obtained from the roots of Astragalus membranaceus, wherein the PGRG1 in the composition has a weight average molecular weight of 10 kDa to 50 kDa and a rhamnose-to-galacturonic acid (Rha:GalA) mole percent ratio of at least 0.06 is provided.

In one aspect, a PGRG1-5K composition having a weight average molecular weight range of 40 to 120 kDa is provided, wherein the PGRG1-5K composition is purified from the PGRG1 composition.

In some embodiments, the isolated PGRG1 in the PGRG1 composition or PGRG1-5K composition has a backbone structure of [1,4 and 1,2-linked RhaGalA]ₘ interspersed with [1,4-linked GalAGalA]ₙ.

In some embodiments, the isolated PGRG1 composition or PGRG1-5K composition is isolated from an Astragalus membranaceus variety selected from Astragalus membranaceus (Fisch.) Bge. and Astragalus membranaceus (Fisch.) var. mongholicus (Bge.) Hsiao varieties.

In some embodiments, the PGRG1 composition or PGRG1-5K composition is isolated from the roots of Astragalus membranaceus grown in a province of Peoples' Republic of China selected from Shanxi, Inner Mongholia, Gansu, Hebei and Liaoning.

In some embodiments, the PGRG1 composition or PGRG1-5K composition is isolated from cultivated Astragalus membranaceus plants that are two years old or younger. In some embodiments, the PGRG1 composition or PGRG1-5K composition is isolated from cultivated Astragalus membranaceus plants that are between two and three years old. In some embodiments, the PGRG1 composition or PGRG1-5K composition is isolated from wild-type Astragalus membranaceus plants that are between around three years old and 10-years old.

In some embodiments, the PGRG1 composition described herein derives from Astragalus species only, for example from Astragalus membranaceus Bge., and does not derive from or include extracts of Dankuei such as polysaccharides from Angelica sinensis Diels, Angelic archangelica or Levisticum officinale Koch.

In one aspect, an oral PGRG1 formulation is provided, the formulation comprising a therapeutically effective amount of the isolated PGRG1 composition or PGRG1-5K composition and, optionally, a pharmaceutically suitable excipient.

In some embodiments, the composition or formulation is administered in combination with excipients or other plant extracts which may (or may not) enhance pharmaceutical efficacy.

In one aspect, a method of treating kidney disease in a mammal by reducing blood creatinine levels is provided, the method comprising orally administering to the mammal an amount of the isolated PGRG1 composition or PGRG1-5K composition effective to reduce blood creatinine levels as compared to blood creatinine levels in an untreated mammal. In one aspect, a method is provided for inhibiting the progression of Chronic Kidney Disease (CKD) as measured by maintaining or reducing the creatinine blood levels in mammals.

In one aspect, a method of inducing hematopoiesis in a mammal, as measured by increased blood cell counts, is provided, the method comprising orally administering to the mammal an amount of the purified PGRG1 composition or PGRG1-5K composition effective to induce hematopoiesis.

In one aspect, a method to modulate the immune system is provided.

In one aspect, a method of treating immune system dysfunction is provided, the method comprising orally administering to a mammal an amount of the purified PGRG1 composition or PGRG1-5K composition effective to treat immune system dysfunction.

In some embodiments, the mammal is a human.

In some embodiments, the method further comprises administering at least one additional therapeutic agent in combination with the PGRG1 composition or PGRG1-5K composition.

In one aspect, a method of producing the PGRG1 composition or the PGRG1-5K composition is provided, the method comprising extracting the dried roots of Astragalus membranaceus in an aqueous solution at about 100° C. over a total of nine hours, and adding lower alkanol to the extract in an amount sufficient to precipitate the PGRG1 composition, resulting in an isolated precipitated PGRG1 or PGRG1-5K composition.

In one aspect, a method of producing an isolated PGRG1-5K composition is provided, the method comprising subjecting an aqueous solution of the isolated PGRG1 composition to ultrafiltration through a filter having a 5 kDa molecular weight cutoff and isolating the PGRG1-5K composition from the retentate.

In one aspect, a dietary supplement comprising the purified PGRG1 composition or the PGRG1-5K composition is provided.

Additional embodiments of the presently disclosed methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the disclosure are set forth in the following description and claims, particularly when considered in conjunction with the accompanying examples and drawings.

### BRIEF DESCRIPTION OF THE DRAWINGS

FIG. 1 illustrates exemplary sugar residues found in polysaccharides;

FIG. 2 illustrates an exemplary PGRG1 chemical structure;

FIG. 3 shows the molecular weight distributions of PGRG1 and PGRG1-5K, as analyzed by HPLC-RI;

FIGS. 4A-4C show three Weighted Average Molecular Weight (waMW) analyses of PGRG1 by HPLC-RI;

FIGS. 5A-5C show three waMW analyses of PGRG1-5K by HPLC-RI;

FIG. 6 shows the effects of PGRG1 on blood creatinine levels; and

FIGS. 7A-7D show the effects of PGRG1 on platelets, white blood cells, neutrophils and red blood cells, respectively.

### DETAILED DESCRIPTION

Various aspects and embodiments of the present disclosure are set forth and described more fully hereinbelow. Such aspects are meant to be exemplary and illustrative, not limiting in scope, and may be embodied in many different forms; these aspects and embodiments are not to be construed as limited to those explicitly set forth herein. Rather, these aspects and embodiments are provided so that this disclosure will be thorough and complete, and will fully convey its scope to those skilled in the art.

The practice of the present disclosure will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current edition); Morrison and Boyd, Organic Chemistry (Allyn and Bacon, Inc., current edition); J. March, Advanced Organic Chemistry (McGraw Hill, current edition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed. “Synthesis of peptides and peptidomimetics” Methods of organic chemistry (Houben-Weyl): additional and supplementary volumes to the 4th edition, 2004, Goodman, Murray; Toniolo, Claudio; Moroder, Luis; Felix, Aurthur; Thieme Medical Publishers Inc.

### I. Definitions

Unless defined otherwise, all technical and scientific terms used herein have the same meanings as commonly understood by one of ordinary skill in the art in the field. Any methods, devices and materials similar or equivalent to those described herein can be used in the practice or testing of the present invention. All publications mentioned herein are incorporated herein by reference for the purpose of describing and disclosing the methodologies and materials reported in the publications which might be used in connection with the present disclosure.

As used in this specification and in the appended claims, the singular forms “a,” “an,” and “the” include plural referents unless the context clearly dictates otherwise. Thus, for example, reference to “a carbohydrate” includes a single carbohydrate molecule as well as two or more of the same or different carbohydrates, reference to “a pectin” includes a single type of pectin as well as two or more of the same or different types of pectins, reference to an “excipient” includes a single excipient as well as two or more of the same or different excipients, and the like.

Where a range of values is provided, it is intended that each intervening value between the upper and lower limit of that range and any other stated or intervening value in that stated range is encompassed within the disclosure; each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed, subject to any specifically excluded limit in the stated range. Where the stated range includes one or both of the limits, ranges excluding either or both of those included limits are also included. For example, if a range of 1% to 8% is stated, it is intended that 2%, 3%, 4%, 5%, 6%, and 7% are also explicitly disclosed, as well as the range of values greater than or equal to 1% and the range of values less than or equal to 8%. Similarly, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

Provided herein is an isolated polygalacturonan rhamnogalacturonan 1 (PGRG1) composition obtained from the roots of Astragalus membranaceus, wherein the PGRG1 in the composition has a weight average molecular weight of 10 kDa to 50 kDa and a rhamnose-to-galacturonic acid (Rha:GalA) mole percent ratio of at least 0.06. In other words, the PGRG1 in the composition occurs in a ratio of at least 0.06 rhamnose-to-galacturonic acid as measured in mole percent.

Also provided is a PGRG1-5K composition purified from the PGRG1 composition and having a weight average molecular weight range of 40 to 120 kDa.

In some embodiments, the isolated PGRG1 composition or PGRG1-5K composition is isolated from an Astragalus membranaceus variety selected from Astragalus membranaceus (Fisch.) Bge. and Astragalus membranaceus (Fisch.) var. mongholicus (Bge.) Hsiao species.

In some embodiments, the PGRG1 composition or PGRG1-5K composition is isolated from Astragalus membranaceus grown in a province of Peoples' Republic of China selected from Shanxi, Inner Mongholia, Gansu, Hebei and Liaoning.

In some embodiments, the PGRG1 composition or PGRG1-5K composition is isolated from cultivated Astragalus membranaceus plants that are two years old or younger. In some embodiments, the PGRG1 composition or PGRG1-5K composition is isolated from cultivated Astragalus membranaceus plants that are between two and three years old. In some embodiments, the PGRG1 composition or PGRG1-5K composition is isolated from wild-type Astragalus membranaceus plants that are between around three years old and 10-years old.

In some embodiments, the PGRG1 in the PGRG1 composition or PGRG1-5K composition has a backbone structure of [1,4 and 1,2-linked RhaGalA]ₘ interspersed with [1,4-linked GalAGalA]ₙ.

Also provided is an oral PGRG1 formulation comprising a therapeutically effective amount of the isolated PGRG1 composition or PGRG1-5K composition and, optionally, a pharmaceutically suitable excipient.

Also provided is a method of producing the PGRG1 composition, comprising (a) extracting the dried roots of Astragalus membranaceus in an aqueous solution at about 100° C. over a total of nine hours, and adding to the extract sufficient lower alkanol to precipitate the PGRG1 composition, resulting in an isolated PGRG1 composition.

Also provided is a method of producing the PGRG1-5K composition, the method comprising subjecting an aqueous solution of the PGRG1 composition to ultrafiltration through a filter having a 5 kDa molecular weight cutoff, and isolating the PGRG1-5K composition from the retentate.

Also provided is a method of treating kidney disease in a mammal by reducing blood creatinine levels, comprising orally administering to the mammal the PGRG1 composition, the PGRG1-5K composition, or an oral formulation thereof, an amount effective to reduce blood creatinine levels as compared to blood creatinine levels in an untreated mammal.

Also provided is a method of inducing hematopoiesis in a mammal, as measured by increased blood cell counts, by orally administering to the mammal the PGRG1 composition, the PGRG1-5K composition, or an oral formulation thereof, an amount effective to induce hematopoiesis.

Also provided is a method of treating immune system dysfunction by orally administering to a mammal the PGRG1 composition, the PGRG1-5K composition, or an oral formulation thereof, an amount effective to treat immune system dysfunction.

In some embodiments, the mammal is a human.

In some embodiments, the method further comprises administering at least one additional therapeutic agent.

Also provided is a dietary supplement comprising the purified PGRG1 composition or the PGRG1-5K composition.

In some embodiments, the variety is Astragalus membranaceus. In some embodiments, the plant tissue used to prepare the composition is taken exclusively from the root of the plant. In some embodiments, the backbone structure is [1,4 and 1,2-linked RhaGalA]ₘ interspersed with [1,4-linked GalAGalA]ₙ. In some embodiments, the biological activity is hematopoiesis and kidney protection. In some embodiments, the PGRG1 is purified by 5 k dialysis.

As used herein, the following terms are intended to have the following meanings:

The phrase “nucleic acid sequence” (or nucleic acid molecule) refers to a DNA or RNA molecule in single or double stranded form, particularly a DNA encoding a protein or protein fragment according to the present disclosure. An “isolated nucleic acid sequence” refers to a nucleic acid sequence which is no longer in the natural environment from which it was isolated, e.g. the nucleic acid sequence in a bacterial host cell or in the plant nuclear or plastid genome.

The terms “protein” or “polypeptide” are used interchangeably and refer to molecules consisting of a chain of amino acids, without reference to a specific mode of action, size, three-dimensional structure or origin. Thus, a “fragment” or “portion” of a protein may still be referred to as a “protein” or may be referred to as a “polypeptide” or a “peptide.” An “isolated protein” is used to refer to a protein which is no longer in its natural environment, for example in vitro or in a recombinant bacterial or plant host cell. An enzyme is a protein comprising enzymatic activity.

In some embodiments, the PGRG1 or PGRG1-5K composition described herein is at least partially purified from an extract obtained from leaves, stems and/or roots of Astragalus membranaceus.

The terms “purified,” “substantially purified,” and “isolated,” as used herein, refer to the state of being free of other, dissimilar compounds with which the PGRG1 or PGRG1-5K is normally associated in its natural state. Preferably, “purified,” “substantially purified,” and “isolated” mean that the composition comprises at least 0.5%, 1%, 5%, 10%, 20%, 30% or 40%, at least 50%, at least 60%, at least 70%, at least 75%, at least 80%, at least 85%, or at least 90% of the mass, by weight, of a given sample. In some embodiments, these terms refer to compositions in which the PGRG1 or PGRG1-5K comprises at least 95% of the mass, by weight, of a given sample. As used herein, the terms “purified,” “substantially purified,” and “isolated,” when referring to a PGRG1 or PGRG1-5K composition, also refers to a state of purification or concentration different than that which may occur naturally in the botanical source. Any degree of purification or concentration greater than that which occurs naturally in the source, including (1) the purification away from other associated structures or compounds or (2) the association with structures or compounds to which it is not normally associated in the botanical source, are within the meaning of “isolated.” The PGRG1 or PGRG1-5K compositions described herein may be isolated or otherwise associated with structures or compounds to which they are not normally associated in nature, according to a variety of methods and processes known to those of skill in the art.

Further, the term “substantially purified,” as used herein, refers to PGRG1 or PGRG1-5K compositions that are removed from their natural environment, isolated or separated, and are at least 60% free, at times 75% free, at times 90% free, and at times 95% free from other components with which they may be otherwise naturally associated or have become associated during the purification process.

In some embodiments, the PGRG1 or PGRG1-5K composition is at least partially purified or liberated from other plant components by treatment with heat, light, pH change, autoxidation, a shift of equilibrium in solution, or a change in concentration or ionic strength, for example. Thus, the PGRG1 or PGRG1-5K in the composition described herein may be chemically or thermally modified as compared to the original PGRG1 or PGRG1-5K in its naturally occurring botanical context.

Similar to proteins, primary, secondary, tertiary and quarternary levels of structural organization exist for polysaccharides; thus, polysaccharides can be described at the level of molecules, clusters (aggregates), networks or interactions. The three-dimensional molecular structural analysis of polysaccharides is a burgeoning field of research. Furthermore, a polysaccharide can be described by (1) its primary structure, i.e., the covalent sequence of monosaccharide residues in the polymer chain; (2) its secondary “ordered” structure, which defines any geometrically regular arrangement in space that the primary sequence may adopt; (3) its tertiary structure, which defines the way second order arrangements pack together; and (4) higher quaternary structures, which define polysaccharide-polysaccharide interactions. Polysaccharide clusters and gel networks have been observed for carrageenan, alginate, pectin, and gellan. For the synergistic interactions of galactomannans or konjac glucomannan with helix-forming algal polysaccharides or xanthan, higher levels of structural organization (polysaccharide-polysaccharide interactions) have been elucidated. (Eggleston and Doyle, “Advances in Biopolymers: Molecules, Clusters, Networks, and Interactions,” Chapter 2, pp 19-34, Volume 935, published Aug. 28, 2006. Eds. Marshall L. Fishman, Phoebe X. Qi, and Louise Wicker).

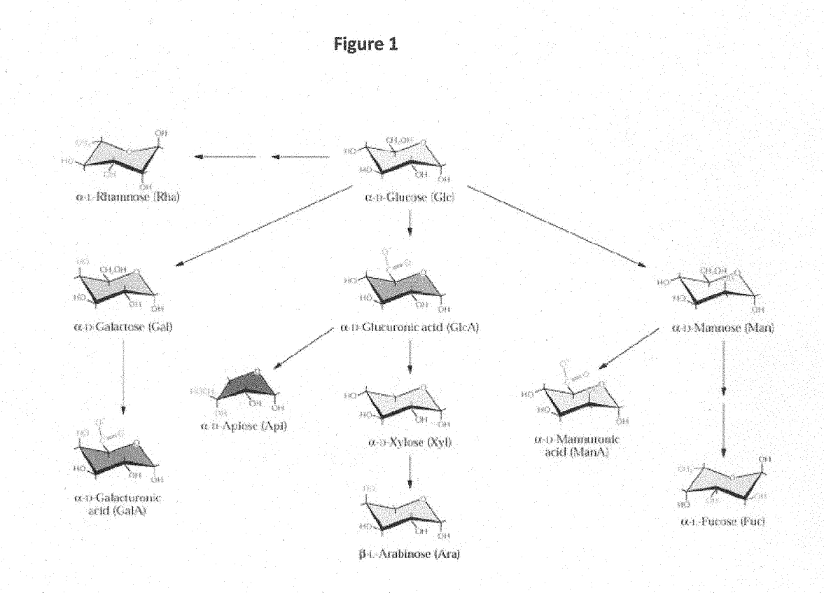

Exemplary sugar residues identified herein are: arabinosyl (Ara), rhamnosyl (Rha), galacturonic acid (GalA), galactosyl (Gal), and glucosyl (Glc) residues. [FIG. 1].

The term “pharmaceutical grade” means that certain specified biologically active and/or inactive components in a botanical drug must be within certain specified absolute and/or relative concentration range and/or that the components must exhibit certain activity levels as measured by a disease-, disorder or condition-specific bioactivity assay. The disease, disorder or condition may afflict a human or an animal.

As used herein, “components” means discrete compounds (i.e. chemicals) which either are present naturally in a botanical composition or have been added to the botanical drug so as to prepare a pharmaceutical grade botanical drug having components within a defined bioactivity range(s) and/or compositional ranges.

“Active components(s)” means one or more component(s) for which the summation of the individual component(s) activity in a disease-specific bioassay accounts for a substantial portion of the observed biological activity of botanical material. In some embodiments, the summation of the active components' activities accounts of the majority (greater than 50%) of the observed biological activity.

As used herein, “fractions” typically means a group of components or class of structurally similar components having defined parameters such as solubility, molecular weight range, polarity range, adsorption coefficients, binding characteristics, chemical reactivity or selective solubility. Most frequently, fractions will be the product of chromatographic separation techniques, i.e., flash chromatography, preparative high performance liquid chromatography (HPLC), preparative gas chromatography, preparative thin layer chromatography, affinity chromatography, size exclusion chromatography, liquid-liquid chromatography e.g., counter-current chromatography or centripetal chromatography.

Acetogenins, alkaloids, carbohydrates, carotenoids, cinnamic acid derivatives, fatty acids, fatty acid esters, flavonoids, glycosides, isoprenoids, macrocyclic antibiotics, nucleic acids, penicillins, peptides, phenolics, polyacetylenes, polyketides, polyphenols, polysaccharides, proteins, prostaglandins, steroids and terpenoids, alone or in combination, may be active component(s) of the PGRG1 compositions described herein.

In these methods, the aliquot may be separated into both biologically active and inactive components. Furthermore, the fractions may comprise a class of related components.

“Weight average molecular weight (waMW)” is based on a weight distribution of the polymers (i.e., polysaccharides) in a composition. First, the number average molecular weight is calculated, which is defined as the total weight of the molecules in the sample divided by the total number of molecules in the sample: ΣNᵢMᵢ/ΣNᵢ, where Nᵢ is the number of molecules and Mᵢ is the mass of each molecule. Next, the weight average molecular weight (waMW) is calculated from the weight fraction of each type of molecule (where Wᵢ represents the fraction of the total weight represented by each type of molecule). Thus, the weight average molecular weight (waMW) equals ΣWᵢMᵢ.

The bioactivity/clinical indication for the botanical may be associated with any disease, disorder or condition of humans or other animals. Thus, the methods are useful to produce pharmaceutical grade botanical drugs for treatment and/or amelioration and/or prevention of human and/or veterinary diseases, disorders or conditions. Exemplary indications include, but are not limited to, an allergic/inflammatory disorder, a cardiovascular disorder, a cancer or a central nervous system disorder, a gastrointestinal disorder, a metabolic disorder, nausea or a disorder induced by a microbial organism or a virus.

“Mammal” includes humans and non-human mammals, such as companion animals (cats, dogs, and the like) and farm animals (cattle, horses, sheep, goats, swine, and the like).

“Morbidity” refers to conditions, such as diseases or disorders that compromise the health and well-being of an organism, such as an animal. Morbidity-susceptibility or morbidity-associated genes are genes that, when altered, for example, by a variation in nucleotide sequence, facilitate the expression of a specific disease clinical phenotype. Thus, morbidity susceptibility genes have the potential, upon alteration, of increasing the likelihood or general risk that an organism will develop a specific disease.

“Mortality” refers to the statistical likelihood that an organism, particularly an animal, will not survive a full predicted lifespan. Hence, a trait or a marker, such as a polymorphism, associated with increased mortality is observed at a lower frequency in older than younger segments of a population.

“Disease” includes any unhealthy condition of an animal, including a condition detrimental to health resulting from medical therapy (a “side-effect”), and can include autoimmune diseases and conditions of the internal organs, such as, for example the kidney.

“Predisposition to develop a disease or disorder” means that a subject having a particular genotype and/or haplotype has a higher likelihood than one not having such a genotype and/or haplotype for developing a particular disease or disorder.

“Ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

A “therapeutically effective amount” means the amount that, when administered to an animal for treating a disease, is sufficient to effect treatment for that disease.

The active compound(s) and composition(s) of the present disclosure will generally be used in an amount effective to treat or prevent the particular disease being treated. The composition may be administered therapeutically to achieve therapeutic benefit or prophylactically to achieve prophylactic benefit. By therapeutic benefit is meant eradication or amelioration of the underlying disorder being treated, e.g., eradication or amelioration of the underlying disease, disorder or allergy, and/or eradication or amelioration of one or more of the symptoms associated with the underlying disease, disorder or allergy, such that the patient reports an improvement in feeling or condition, notwithstanding that the patient may still be afflicted with the underlying disorder. For example, administration of an active compound to a patient suffering from an allergy provides therapeutic benefit not only when the underlying allergic response is eradicated or ameliorated, but also when the patient reports a decrease in the severity or duration of the symptoms associated with the allergy following exposure to the allergen. Therapeutic benefit also includes halting or slowing the progression of the condition, disorder, disease or allergy, regardless of whether improvement is realized.

The amount of active compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art. Initial dosages may be estimated initially from in vitro assays. Initial dosages can also be estimated from in vivo data, such as animal models.

The amount of extract in the compositions can vary according to factors such as type of disease, age, sex, and weight of the subject. Dosage regimens may be adjusted to optimize a therapeutic response. In some embodiments, a single bolus may be administered; several divided doses may be administered over time; the dose may be proportionally reduced or increased; or any combination thereof, as indicated by the exigencies of the therapeutic situation and factors known one of skill in the art. It is to be noted that dosage values may vary with the severity of the condition to be alleviated. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners.

The terms “administration” or “administering” refer to a method of incorporating a composition into the cells or tissues of a subject, either in vivo or ex vivo to diagnose, prevent, treat, or ameliorate a symptom of a disease. In one example, a compound can be administered to a subject in vivo parenterally. In another example, a compound can be administered to a subject by combining the compound with cell tissue from the subject ex vivo for purposes that include, but are not limited to, assays for determining utility and efficacy of a composition. When the compound is incorporated in the subject in combination with one or active agents, the terms “administration” or “administering” can include sequential or concurrent incorporation of the compound with the other agents such as, for example, any agent described above. A pharmaceutical composition of the present disclosure is formulated to be compatible with its intended route of administration. Examples of routes of administration include, but are not limited to, parenteral such as, for example, intravenous, intradermal, intramuscular, and subcutaneous injection; oral; inhalation; intranasal; transdermal; transmucosal; and rectal administration.

An “effective amount” of a compound of the present disclosure can be used to describe a therapeutically effective amount or a prophylactically effective amount. An effective amount can also be an amount that ameliorates the symptoms of a disease. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount may need to be administered in an amount sufficient to result in amelioration of one or more symptoms of a disorder, prevention of the advancement of a disorder, or regression of a disorder. In one example, treatment of an inflammatory disorder or an autoimmune disorder characterized by inflammation, a therapeutically effective amount preferably refers to the amount of a therapeutic agent that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the composition. The term “treating” refers to the administering one or more therapeutic or prophylactic agents taught herein.

A “prophylactically effective amount” refers to an amount that is effective at the dosages and periods of time necessary to achieve a desired prophylactic result such as, preventing or inhibiting the severity of a platelet or white blood cell count drop, or reducing the nadir of the drop. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

In some embodiments, the administration can be oral. In other embodiments, the administration can be subcutaneous injection. In some embodiments, the administration can be intravenous injection using a sterile isotonic aqueous buffer. In some embodiments, the administration can include a solubilizing agent and a local anesthetic such as lignocaine to ease discomfort at the site of injection. In other embodiments, the administrations may be parenteral to obtain, for example, ease and uniformity of administration.

In some embodiments, a therapeutically or prophylactically effective amount of a composition may range in concentration from about 0.001 nM to about 0.10 M; from about 0.001 nM to about 0.5 M; from about 0.01 nM to about 150 μM; from about 0.01 nM to about 500 μM; from about 0.01 nM to about 1000 μM, or any range therein. In some embodiments, the compositions may be administered in an amount ranging from about 0.001 mg/kg to about 500 mg/kg; from about 0.005 mg/kg to about 400 mg/kg; from about 0.01 mg/kg to about 300 mg/kg; from about 0.01 mg/kg to about 250 mg/kg; from about 0.1 mg/kg to about 200 mg/kg; from about 0.2 mg/kg to about 150 mg/kg; from about 0.4 mg/kg to about 120 mg/kg; from about 0.15 mg/kg to about 100 mg/kg, from about 0.15 mg/kg to about 50 mg/kg, from about 0.5 mg/kg to about 10 mg/kg, or any range therein, wherein a human subject is assumed to average about 70 kg.

Dosage amounts will typically be in the range of from about 1 mg/kg/day to about 100 mg/kg/day, 200 mg/kg/day, 300 mg/kg/day, 400 mg/kg/day or 500 mg/kg/day, but may be higher or lower, depending upon, among other factors, the activity of the active compound, its bioavailability, the mode of administration and various factors discussed above. Dosage amount and interval may be adjusted individually to provide plasma levels of the active compound(s) which are sufficient to maintain therapeutic or prophylactic effect. In cases of local administration or selective uptake, such as local topical administration, the effective local concentration of active compound(s) may not be related to plasma concentration. Skilled artisans will be able to optimize effective local dosages without undue experimentation.

The compounds can be administered in dosage units. The term “dosage unit” refers to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

The compound(s) may be administered once per day, a few or several times per day, or even multiple times per day, depending upon, among other things, the indication being treated and the judgment of the prescribing physician.

Preferably, the active compound(s) will provide therapeutic or prophylactic benefit without causing substantial toxicity. Toxicity of the active compound(s) may be determined using standard pharmaceutical procedures. The dose ratio between toxic and therapeutic (or prophylactic) effect is the therapeutic index. Active compound(s) that exhibit high therapeutic indices are preferred.

“Transmucosal” or similar terms means passage of a permeant into and through the mucosa to achieve effective therapeutic blood levels or deep tissue levels of a drug.

“Chemical enhancer,” “penetration enhancer,” “permeation enhancer,” and the like shall be inclusive of all enhancers which increase the flux of a permeant, drug, or other molecule across the mucosa and is limited only by functionality. In other words, all cell envelope disordering compounds, solvents, steroidal detergents, bile salts, chelators, surfactants, non-surfactants, fatty acids, and any other chemical enhancement agents are intended to be included. The flux of a drug or analyte across the mucosa can be increased by changing either the resistance (the diffusion coefficient) or the driving force (the gradient for diffusion). Flux may be enhanced by the use of so-called penetration or permeation or chemical enhancers.

Permeation enhancers are comprised of two primary categories of components, i.e., cell-envelope disordering compounds and solvents or binary systems containing both cell-envelope disordering compounds and solvents. As discussed above, other categories of permeation enhancer are known, however, such as steroidal detergents, bile salts, chelators, surfactants, non-surfactants, and fatty acids.

Combinations of enhancers consisting of diethylene glycol monoethyl or monomethyl ether with propylene glycol monolaurate and methyl laurate are disclosed in U.S. Pat. No. 4,973,468 as enhancing the transdermal delivery of steroids such as progestogens and estrogens. A dual enhancer consisting of glycerol monolaurate and ethanol for the transdermal delivery of drugs is shown in U.S. Pat. No. 4,820,720. U.S. Pat. No. 5,006,342 lists numerous enhancers for transdermal drug administration consisting of fatty acid esters or fatty alcohol ethers of C2 to C4 alkanediols, where each fatty acid/alcohol portion of the ester/ether is of about 8 to 22 carbon atoms. U.S. Pat. No. 4,863,970 shows penetration-enhancing compositions for topical application comprising an active permeant contained in a penetration-enhancing vehicle containing specified amounts of one or more cell-envelope disordering compounds such as oleic acid, oleyl alcohol, and glycerol esters of oleic acid; a C2 or C3 alkanol and an inert diluent such as water.

Other permeation enhancers, not necessarily associated with binary systems include DMSO or aqueous solutions of DMSO such as taught in Herschler, U.S. Pat. No. 3,551,554; Herschler, U.S. Pat. No. 3,711,602; and Herschler, U.S. Pat. No. 3,711,606, and the azones (n-substituted-alkyl-azacycloalkyl-2-ones) such as noted in Cooper, U.S. Pat. No. 4,557,943.

“Permeant,” “drug,” or “pharmacologically active agent” or any other similar term means any chemical or biological material or compound, inclusive of peptides, suitable for transmucosal administration by the methods previously known in the art and/or by the methods taught in the present disclosure, that induces a desired biological or pharmacological effect, which may include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect may be local, such as providing for a local anaesthetic effect, or it may be systemic. The present disclosure is not drawn to novel permeants or active agents. Rather it is limited to agents or permeants which exist in the state of the art or which may later be established as active agents for use in combination with the compounds and compositions of the present disclosure. Such substances include broad classes of compounds normally delivered into the body, including through body surfaces and membranes, including skin. In general, this includes but is not limited to: antiinfectives such as antibiotics and antiviral agents; analgesics and analgesic combinations; anorexics; antihelminthics; antiarthritics; antiasthmatic agents; anticonvulsants; antidepressants; Antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychostimulants; sedatives; and tranquilizers. By the method of the present disclosure, both ionized and nonionized drugs may be delivered, as can drugs of either high or low molecular weight.

“Buccal” drug delivery is meant delivery of a drug by passage of a drug through the buccal mucosa into the bloodstream. Preferably, buccal drug delivery is affected herein by placing the buccal dosage unit on the upper gum or opposing inner lip area of the individual undergoing drug therapy.

“Excipients” or “vehicles” as used herein refer to any excipients or vehicles suitable for oral or buccal drug administration, and include any such materials known in the art, e.g., any liquid, gel, solvent, liquid diluent, solubilizer, or the like, which is nontoxic and which does not interact with other components of the composition in a deleterious manner.

“Pharmaceutically acceptable excipient” means a diluent, adjuvant, excipient or vehicle that is useful in preparing and/or administering a pharmaceutical composition that is generally safe, non-toxic, and desirable, and includes excipients that are acceptable for human pharmaceutical use as well as for veterinary use. Such excipients may be solid, liquid, semisolid, or, in the case of an aerosol composition, gaseous. These pharmaceutical carriers include any and all physiologically compatible solvents, dispersion media, coatings, antibacterial and antifungal agents, isotonic and absorption delaying agents, and the like.

Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Water can be a preferred carrier for intravenous administration. Saline solutions, aqueous dextrose and glycerol solutions can also be liquid carriers, particularly for injectable solutions. A carrier is pharmaceutically acceptable after approval by a state or federal regulatory agency or listing in the U.S. Pharmacopeial Convention or other generally recognized sources for use in subjects.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof.

The compositions can take the form of solutions, suspensions, emulsion, tablets, pills, capsules, powders, sustained-release formulations and the like. Oral formulations can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences. Supplementary active compounds can also be incorporated into the compositions. In some embodiments, the carrier is suitable for parenteral administration. In other embodiments, the carrier can be suitable for intravenous, intraperitoneal, intramuscular, sublingual or oral administration. In other embodiments, the pharmaceutically acceptable carrier may comprise pharmaceutically acceptable salts.

Pharmaceutical formulations for parenteral administration may include liposomes. Liposomes and emulsions are delivery vehicles or carriers that are especially useful for hydrophobic drugs. Depending on biological stability of the therapeutic reagent, additional strategies for protein stabilization may be employed. Furthermore, one may administer the drug in a targeted drug delivery system such as, for example, in a liposome coated with target-specific antibody. The liposomes will bind to the target protein and be taken up selectively by the cell expressing the target protein.

Therapeutic compositions typically must be sterile and stable under the conditions of manufacture and storage. The composition can be formulated as a solution, microemulsion, liposome, or other ordered structure suitable for a high drug concentration. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

In some embodiments, isotonic agents can be used such as, for example, sugars; polyalcohols including, but not limited to, mannitol, sorbitol, glycerol, and combinations thereof and sodium chloride. Sustained absorption characteristics can be introduced into the compositions by including agents that delay absorption such as, for example, monostearate salts, gelatin, and slow release polymers. Carriers can be used to protect active compounds against rapid release, and such carriers include, but are not limited to, controlled release formulations in implants and microencapsulated delivery systems. Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer (PLG), and the like. Such formulations can generally be prepared using methods known to one of skill in the art.

The compounds may be administered as suspensions such as, for example, oily suspensions for injection. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions.

In some embodiments, a sterile and injectable solution can be prepared by incorporating an effective amount of an active compound in a solvent with any one or any combination of desired additional ingredients described above, filtering, and then sterilizing the solution. In another embodiment, dispersions can be prepared by incorporating an active compound into a sterile vehicle containing a dispersion medium and any one or any combination of desired additional ingredients described above. Sterile powders can be prepared for use in sterile and injectable solutions by vacuum drying, freeze-drying, or a combination thereof, to yield a powder that can be comprised of the active ingredient and any desired additional ingredients. Moreover, the additional ingredients can be from a separately prepared sterile and filtered solution. In another embodiment, the extract may be prepared in combination with one or more additional compounds that enhance the solubility of the extract.

In some embodiments, the compounds can be administered by inhalation through an aerosol spray or a nebulizer that may include a suitable propellant such as, for example, dichlorodifluoromethane, trichlorofluoromethane, dichlorotetrafluoroethane, carbon dioxide, or a combination thereof. In one example, a dosage unit for a pressurized aerosol may be delivered through a metering valve. In another embodiment, capsules and cartridges of gelatin, for example, may be used in an inhaler and can be formulated to contain a powderized mix of the compound with a suitable powder base such as, for example, starch or lactose.

A “dietary supplement” means a product intended to supplement the diet that contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any of the aforementioned ingredients.

“Lower alkanol” means refers to an alcohol having one to six carbon atoms. Examples of lower alkanols are methanol, ethanol, butanol, and isopropanol.

In some embodiments, the lower alkanol can be added to the solvent to a concentration of about 70% lower alkanol at about room temperature to create the precipitate. In some embodiments, the precipitation is done by first using a lower concentration of about 35% lower alkanol in a first precipitation step, and then using a higher concentration of about 70% lower alkanol in a second precipitation step. The concentrations of lower alkanol used in the precipitations can range from, for example, 20%-30%, 30%-40%, 40%-50%, 50%-60%, 60%-70%, 70%-80%, 80%-90%, or any concentration therein.

The evolution of plant life is based on constantly occurring genetic mutations, and often a particular individual of a species will mutate to a different color, size, or growth habit. Genetic mutations and their phenotypes are passed on to progeny (offspring). When this happens in plants, a population can exist with the same scientific name, but a sub-group can exhibit varying characteristics. If the mutated sub-group is significantly different from the parents and is stable (the traits are passed on from generation to generation), then this new group of plants is often assigned a variety name. Variety names are given when the mutation occurs in nature.

Thus, a “variety” (“var.”) of an Astragalus species refers to a particular Astragalus membranaceus subgroup, strain, mutation or cultivar. Exemplary varieties of Astragalus membranaceus are: Astragalus membranaceus (Fisch.) Bunge; Astragalus membranaceus (Fisch.) Bunge var. mongholicus (Bunge) P. K. Hsiao; Astragalus propinquus Schischkin; Astragalus propinquus Schischkin var. glabra Vydr.; and Phaca membranacea Fisch. Similarly, cultivar names are given when the mutation occurs due to human influence, and cultivar name is written after cv. or within the single quotes, for example: Pinguicula moranensis cv. Superba or Pinguicula moranensis ‘Superba’.

In some embodiments, the PGRG1 composition described herein derives from Astragalus species only, for example from Astragalus membranaceus Bge., and does not derive from or include extracts of Dankuei such as polysaccharides from Angelica sinensis Diels, Angelic archangelica or Levisticum officinale Koch.

A “modification” of polysaccharide or a sugar residue refers to a methylesterified and/or acetylated polysaccharide or sugar, for example. The polysaccharide or a sugar residue may be naturally occurring, or may be a synthetic analog.

“Pectins” are a complex group of branched heteropolysaccharides abundant in primary cell walls and in the middle lamella between all plant cells. Pectin polymers are chemically diverse acidic molecules containing a high proportion of D-galacturonic acid residues joined by α-1,4 glycosidic linkages. This pectin α-(1-4) polygalacturonic acid backbone and can be randomly acetylated and methylated. Pectins are composed of three main polysaccharides: polygalacturonan, rhamnogalacturonan I (RG-I) and rhamnogalacturonan II (RG-II). Some of the carboxylic acids of the galacturonates are esterified to methanol. L-rhamnose (a 6-deoxyhexose) residues are usually interspersed throughout the chain. The linkage of D-galacturonic acid to L-rhamnose is α-1,2 and the linkage from D-galacturonic acid to the next galacturonic acid in the chain is α-1,4. Side chains of neutral sugar polymers containing monomers such as L-arabinose or D-galactose are often attached to the rhamnose residues. Thus, the RG-I backbone (rhamnosyl and D-galacturonosyl) sections contain “hairy” branch points with side chains of arabinose and galactose (Bacic, 2006, Proc. Natl. Acad. Sci. 102(15):5639-5640).

Pectin is used in the animal feed industry and in the food industry as a gelling agent. In ruminants, bacterial and fungal enzymes in the digestive tract aid its digestion. There is commercial interest in pectin-degrading enzymes to convert the pectin-rich by-products from citrus peel and sugar beet processing to higher value material. Pectinolytic enzymes such as esterases, hydrolases and lyases, are also used in other industries that require the processing of pectin.

In natural pectin the majority of the carboxyl groups of D-galacturonate are methyl esters. Some plants also contain acetylated galacturonate residues. Non-esterified carboxyl groups may be linked through divalent cations such as Mg²⁺ or Ca²⁺, causing pectin to form a gel.

An example of Rhamnogalacturonan I (RG-I) is shown below:

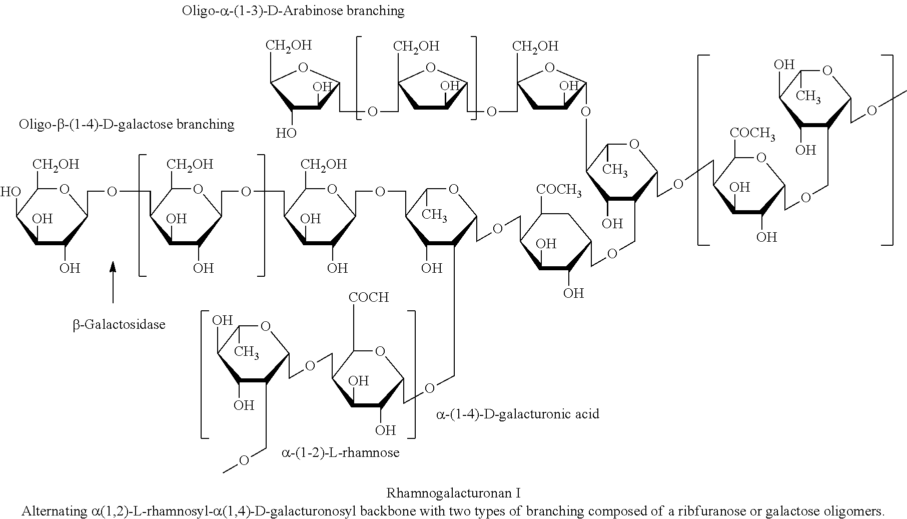

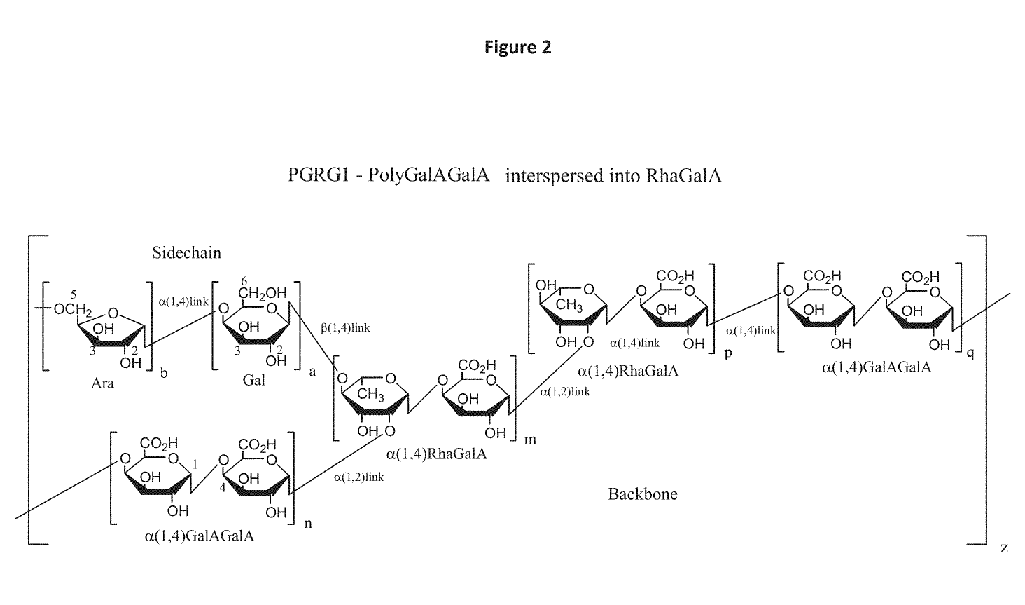

A generic structure of the PGRG1 of the herein disclosed compositions is illustrated in FIG. 2, and is shown below.

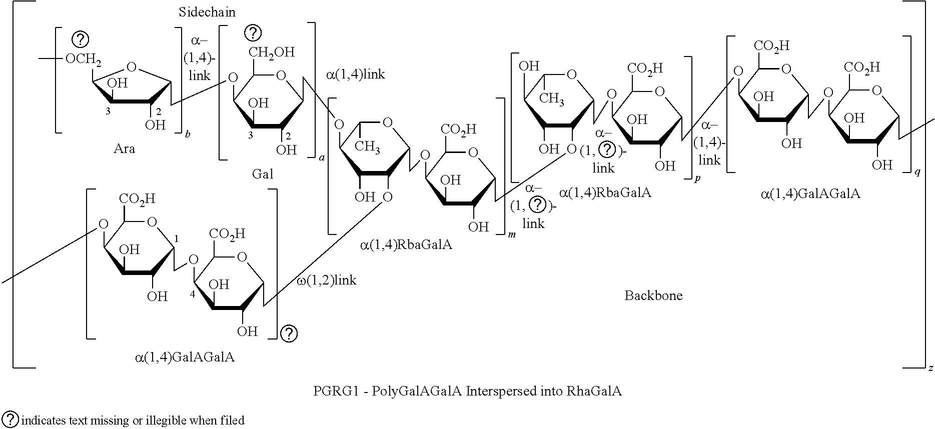

In PGRG1 and PGRG-5K, polyGalAGalA are interspersed into a RhaGalA1 polymer. From FIG. 2, it can be observed that PGRG1 is a polysaccharide made up of disaccharides—Rhamnose Galacturonic Acid (RhaGalA) and Galacturonic Acid Galacturonic Acid (GalAGalA) in the backbone and side chains. The backbone sugar monomers are in the pyranose form. The individual sugars in each of the disaccharides are stereo- and regiochemically linked via alpha 1,4 covalent bond. Individual backbone disaccharides-to-disaccharide linkages can be either alpha 1,2 linked or alpha 1,4 linked. The side chains in PGRG1 are made up monosaccharides—Galactose (Gal) and Arabinose (Ara). The Gal and Ara are in the pyranose and furanose forms, respectively. The Gal is beta (1,4) linked to the Rha in RhaGalA backbone and Ara is alpha(1,4)linked to Gal. There can be significant branching in the side chain. The Gal branching can be 2, 3, and/or 6 linked, and the Ara can be 2, 3 and/or 5 linked.

In the PGRG1 structure shown in FIG. 2 and above, the subscripts n, m, p, q, a, b, and z can be any integer whose resulting structure when converted to a molecular weight falls within the range of 10-50 kDa. For PGRG1-5K, the subscripts n, m, p, q, a, b, and z can be any integer whose resulting structure when converted to a molecular weight falls within the range of 40-120 kDa. For both PGRG1 and PGRG1-5K, the integers n, m, p, q, a, and b can equal 0 with the proviso that no two sequential integers can equal 0.

It will be clear to the skilled artisan that previously described, art-known polysaccharide, pectin and Rhamnogalacturonan I compositions differ significantly from the PGRG1 and PGRG1-5K compositions described herein. For example, art-known compositions are almost always derived from a multitude of species of plants, which have been grown for indefinite periods of time and/or uncontrolled conditions, and harvested after various unspecified growing times, leading to unpredicted variations in chemical composition. Furthermore, art-known compositions are commonly isolated from both the roots and the leaves of plants. The presently disclosed PGRG1 compositions are isolated from roots of Astragalus membranaceus, and have a weight average molecular weight of at least 40 kiloDaltons (kDa).

### ILLUSTRATIVE PUBLICATIONS (INCORPORATED BY REFERENCE HEREIN, EACH IN ITS ENTIRETY)

Disclosed in U.S. Pat. No. 8,137,710; U.S. Patent Application Publications 20120141412 and 20100158861; and PCT Publication WO2010077867 are preparations and uses of compositions comprising extracts of Astragalus membranaceus for treating idiopathic thrombocytopenic purpura. The extracts can comprise, for example, a high-yield extract of Astragalus membranaceus, having an arabinose:galactose ratio greater than about 3.5:1; from about 5% to about 10% rhamnose; from about 15% to about 20% galactose; from about 10% to about 20% galacturonic acid; and, from about 10% to about 15% glucose. Some extracts described include an acid-modified arabinogalactan protein composition having an arabinose:galactose ratio ranging from about 3.5:1 to about 5.0:1, from about 5% to about 10% rhamnose, from about 15% to about 20% galactose, and from about 10% to about 15% glucose. The compositions can be used in the treatment of idiopathic thrombocytopenic purpura and the formulation of medicaments for such treatments. Also disclosed are A. membranaceus Bge. var. mongholicus (Bge.) Hsiao or A. membranaceus (Fisch.) Bge., which may be grown in Inner Mongolia or Shanxi province, Peoples' Republic of China. In some embodiments, the roots are from Astragalus membranaceus plants about two years of age. In some embodiments, the composition has a weight-average molecular weight of at least 100 kDa. In some embodiments, the composition has at least 80% by weight carbohydrate and not more than 2% by weight protein. In some embodiments, the composition has an arabinose:galactose ratio ranging from about 4.0:1 to about 5.0:1.

PCT Publication WO 2001000682 and U.S. Pat. No. 6,991,817 disclose arabinogalactan compositions isolated from Astragalus membranaceus, particularly the roots, said compositions having a weight average molecular weight of at least 100 kDa, and capable of reconstitution into aqueous intravenously injectable formulations, which are useful for stimulating hematopoiesis, inducing the proliferation or maturation of megakaryocytes, stimulating the production of IL-1β, IL-6, TNF-α, IFN-γ, GM-CSF, or G-CSF, stimulating the production or action of neutrophils, treating neutropenia, anemia, or thrombocytopenia, accelerating recovery from exposure (e.g. accidental or non-therapeutic exposure, as well as therapeutic exposure) to cytotoxic (e.g., chemical) agents or radiation, treating cachexia, emesis, or drug withdrawal symptoms, or modifying biological responses or protecting hepatic cells in hepatitis B, in a mammal when intravenously administered to the mammal.

PCT Publication WO2010095808 describes a production method for Astragalus membranaceus extract employing enzymolysis, characterized in that the rate of extraction and recovery of insoluble substances is increased by subjecting Astragalus membranaceus to hydrolysis using an amylase and/or cellulase, and a composition produced by the method, for the prevention or alleviation of diabetes or obesity. The Astragalus membranaceus extract produced using enzymolysis is said to be advantageous in reducing blood sugar, cholesterol and triglycerides, and to prevent or alleviate diabetes (including type 2 diabetes), fatty liver, hyperlipidaemia, arteriosclerosis and obesity.

U.S. Patent application publication US2010173026 describes estrogenic extracts of Astragalus membranaceus Fisch. Bge. Var. mongolicus Bge. of the Leguminosae Family, as well as methods of using said extracts to achieve an estrogenic effect, especially in a female human. In some embodiments, the methods include treatment of climacteric symptoms. In some embodiments, the methods include treatment of estrogen receptor positive cancer, such as estrogen responsive breast cancer. In some embodiments, the methods include treatment or prevention of osteoporosis.

U.S. Pat. Nos. 7,498,048; 7,553,501; and 7,604,823 disclose phyto-nutraceutical mixtures including Astragalus membranaceus (among several other plant extracts).

U.S. Pat. No. 7,364,760 discloses methods for the treatment of a disease state or condition associated with an immunodeficiency virus infection in a mammal, said method comprising administering to a mammal in need thereof a therapeutically effective amount of a pharmaceutical composition comprising therapeutically effective amounts of herbal extracts of Radix Gentianae Longdancao (Gentianae longdancao), Fructus Xanthii Sibirici (Xanthii sibirici), Radix Bupleuri (Bupleurum chinense), Radix Astragali (Astragalus membranaceus), and Chrysanthemum Morifolium (Chrysanthemum morifolium), said composition having a therapeutic index of between at least 80 and 400.

U.S. Pat. No. 4,843,067 discloses a polysaccharide-containing pharmaceutical composition huang-qi (extracted from either Astragalus membranaceus Bge. or Astragalus gummifer Labillard) and polysaccharides of Dankuei from Angelica sinensis Diels, Angelic archangelica or Levisticum officinale Koch for increasing immune function. This huang-qi polysaccharide combination of huang-qi and Dankuei polysaccharides is stated to be extractable by water extraction of a powder of the roots and ethanol precipitation.

U.S. Pat. No. 4,944,946 similarly describes a pharmaceutical composition of four active ingredients ((1) Polysaccharide of Wang Qi from Astragalus membranaceus Bge or other species of Astragalus; (2) Banlankensu from among Isatis tinctoria L, I. indigotica Fort or Baphicacanthus cusia Bremek; (3) Yejuhua-flavonoid from Chrysanthemum indicum L; and (4) Guanzhonhsu from among Dryopteris crassirhizoma Nakai, Osmunda japonica Thunb, Lunathyrium acrostichoides ching or Matteuccia stuthiopteris Todaro) for inhibiting viruses and increasing immune function.

European Patent publication EP 0441278 describes polysaccharides (12000 to 500000 daltons) extracted from the gum exudates of Astragalus membranaceus which have immunomodulating properties, and pharmaceutical compositions containing them.

European Patent publication EP 01374881 discloses a pharmaceutical preparation for asthma made from a part or all of the following Chinese herbal medicines: Ma Huang (Ephedra sinica), Xing Ren (Prunus armeniaca), Gan Cao (Glycyrrhiza uralensis licorice), Huang Qin (Scutellaria baicalensis), Huang Lian (Coptis chinesis), Huang Bai (Phellodron chinense), Kuang Dong Hua (Tusilago farfara), Bai Bu (Stemona sessilifolia), Chuan Bei M U or Bei M U (Fritllaria cirrhosa), Di Long (Pheretima aspergillum), Bu Gu Zhi (Psoralea corylifolia), Dang Shen (Codonopsis pilosula), Shan Zha (Crataegrus pinnatifida), Ma Ya (Hordeum vulgara), Shen Qu (Massa fermentata medicalis), Wu Wei Zi (Schisandra chinensis), Shi Gao (Gypsum), Su Zi (Perilla frutescens), Zi Wan (Aster tataricus), Bai Shao (Paeonia lactiflora), Jin Yin Hua (Lonicera japonica), Lian Qiao (Forsythia suspensa), Jing Jie (Schizonepeta tenuifolia), Huang Qi (Astragalus membranaceus), Fang Feng (Ledebouriella divaricata).

PCT Publication WO 2002078722 and European patent EP 1374880 disclose an antineoplastic composition for the treatment of tumors, substantially composed of the Chinese medicinal herbs: Panax ginsing, Poria cocos, Atractylodes macrocephala, Angelic sinensis, Astragalus membranaceus, Curcuma zedoaria, Scutellaria baicalensis, Coptis chinenisis, Phellodron chinense, Glycyrrhiza uralenisis, Crataegus pinatifida, Hordeum vulgare, Salvia miltiorrhiza, Schisandra chinensis, Hedyotis diffusa, Ophiophogon japonicus, Lobelia chinesis lour, Scutellaria barbaba, Massa fermentata medicalis, Bupleurum scorzonerifolium, Pinellia ternata, Citrus reticulara, Melia toosendan, Paeonia lactiflora, Ganoderma lucium and Zingiber officinale.

PCT Publication WO 2009126652 discloses glycan- or carbohydrate-based methods for the rapid identification of biological markers and therapeutic targets, especially glycan-related targets of infectious diseases, cancers, autoimmune diseases, allergies, inflammation, toxicity, obesity and/or other disorders of humans, animals, plants and other organisms.

PCT Publication WO 2000059520 describes herbal compositions and uses for the treatment or prophylaxis of allergic and inflammatory reactions, such as but not limited to respiratory disorders. The composition comprises herbs or botanical or horticultural equivalents of herbs or chemical or functional equivalents of the herbal extract wherein said composition is effective in the prophylaxis or treatment of an allergic or inflammatory response, wherein the effectiveness of the composition is relative to a placebo-controlled trial, wherein said herbs in the composition are selected to: (i) keep airways open; (ii) invigorate the spleen and digestive system; (iii) reinforce the kidney and function of the urinary and reproductive tract; (iv) eliminate inflammation; and (v) promote blood circulation and remove blood stasis, and wherein the herbs in the composition are selected from the list comprising at least one from five groups of herbs wherein the first group comprises: Mahuang, Guizhi, Cangerzi, Xinyi, Bohe, Xixin, Xingren, Chaihu, Yinxingye, Baizi and Fangfeng; the second group comprises: Baizhu, Shanyao, Dangshen, Dazao, Gancao, Huangqi and Huangjing; the third group comprises: Tusizi, Roucongrong, Fuzi, Yinyanghuo, Buguzhi, Dihuang and Xianmao; the fourth group comprises: Huangqin, Wumei and Wuweizi; the fifth group comprises: Taoren, Chuanxiong, Danpi and Chishao.

PCT Publication WO 2006122454 describes a pharmaceutical composition for treating diabetes and preparation method thereof, wherein the pharmaceutical composition is composed of raw material drugs including radix of Rehmannia glutinosa Libosch; radix of Astragalus membranaceus (Fisch.) Bge. var. mongholicus (Bge.) Hsiao, Astragalus membranaceus (Fisch.) Bge; rhizoma of Dioscorea opposita Thunb; radix of Pueraria lobata (Willd.) Ohwi Pueraria thomsonii Benth; radix of Trichosanthes kirilowii Maxim, Trichosanthes rosthornii Harms; stigma of Zea mays L; fructus of Schisandra sphenanthera Rehd. Et Wils. and glibenclamide. The pharmaceutical composition has the functions of nourishing kidney-Yin, benefiting vital energy and promoting the secretion of saliva, and is used in treating diabetes of deficiency in both vital energy and Yin, namely non-insulin-dependent diabetes.

Japanese patent JP01061411 discloses a hair-restoration agent effective in improving the blood circulation, promoting the growth and generation of hair and preventing the falling off of hair, by using ONRI-YAKU (warming drug) consisting of KANKYO (rhizome of Zingiber officinale), KAKKETSU-KYOYO-YAKU (blood activation drug) consisting of KOKA (flower of Carthamus tinctorius), HOKI-YAKU (invigoration drug) consisting of NINJIN (rhizome of Panax ginseng) and OUGI (root of Astragalus membranaceus), HOKETSU-YAKU (hematopoietic drug) consisting of TOM (rot of Angelica acutiloba) and HOYO-YAKU (analeptic drug) consisting of HOKOTSUSHI (bean of Psoralea corylifolia). The hair-restoration drug contains, as essential components, (a) ONRI-YAKU consisting of KANKYO effective in warming the spleen, stomach and heart to promote the blood flow and improve the blood circulation at the bald or sparsely haired part of the scalp, (b) KAKKETSU-KYOYO-YAKU consisting of KOKA, TONIN (stone of Prunus persica) and TANJIN (bark of Schinopsis lorentzii) effective in eliminating and preventing the stagnation of blood flow, (c) HOKI-YAKU consisting of NINJIN and OUGI effective in replenishing the deficiency of vigor, stimulating the generation of blood and invigorating the spleen and stomach, (d) HOKETSU-YAKU consisting of TOM effective in replenishing and activating the blood and (e) HOYO-YAKU consisting of HOKOTSUSHI effective in invigorating and nourishing the energy and vitality, and is effective in stimulating the bald and alopecic part of scalp, warming and activating the spleen and stomach, eliminating the stagnation of blood flow to keep smooth circulation of blood.

Japanese patent JP06070217 discloses polysaccharide and biophylactic activation agent from the aboveground part of A. mongholicus Bunge and A. membranaceus. The biophylactic activation agent contains an effective component comprising a polysaccharide composition selected from arabinose, rhamnose, fucose, xylulose, mannose, galactose, glucose, glucuronic acid and galacturonic acid originating from the aboveground part of Astragalus mongholicus and Astragalus membranaceus.

### Research Publications:

Thirteen polysaccharides isolated from an extract of the aerial portions of Astragalus mongholics Bunge were reported to have immunomodulating activity against Peyer's patch immunocompetent cells. Rhamnogalacturonan I (RG-I) with beta-D-(1->3,6)-galactosyl side-chains having terminal beta-D-GlcA showed activity in the pectin-enriched fractions. Interestingly, the terminal GlcA was not required for activity of the arabinogalactan-enriched fractions, suggesting at least two different immunomodulating structures. (Hiroaki Kiyohara, et al., 2010, “Different contributions of side-chains in beta-D-(1->3,6)-galactans on intestinal Peyer's patch-immunomodulation by polysaccharides from Astragalus mongholics Bunge.”Phytochemistry, 71(2-3):280-293).

Monoclonal antibody CCRC-M7 is representative of a group of antibodies with similar binding specificity generated using RG-I as immunogen. The epitope recognized by CCRC-M7 is present in several plant polysaccharides and membrane glycoproteins. Selective enzymatic or chemical removal of arabinosyl residues from RG-I reduced, but did not abolish, the ability of CCRC-M7 to bind to the polysaccharide. In contrast, enzymatic removal of both arabinosyl and galactosyl residues from RG-I completely abolished binding of CCRC-M7 to the resulting polysaccharide. Competitive ELISAs using chemically defined oligosaccharides to compete for the CCRC-M7 binding site showed that oligosaccharides containing (1→6)-linked beta-D-galactosyl residues were the best competitors among those tested, with the tri-, penta-, and hexa-saccharides being equally effective. The combined results from indirect and competitive ELISAs suggest that the minimal epitope recognized by CCRC-M7 encompasses a (1→6)-linked beta-galactan containing at least three galactosyl residues with at least one arabinosyl residue attached. (Steffan, et al., “Characterization of a monoclonal antibody that recognizes an arabinosylated (1→6)-beta-D-galactan epitope in plant complex carbohydrates.” Carbohydrate Res. 275(2)295-307).

Gum tragacanth samples from six species of Iranian Astragalus “goat's-horn” bush plants (Astragalus parrowianus, Astragalus fluccosus, Astragalus rahensis, Astragalus gossypinus, Astragalus microcephalus, and Astragalus compactus) were evaluated for viscoelastic properties and emulsion stabilizing effects, with respect to their detailed chemical composition in order to examine any possible correlation between the make-up and the emulsion stabilizing properties of gum tragacanth. The monosaccharide make-up of the six gums also varied, but all the gums contained relatively high levels of galacturonic acid (approximately 100-330 mg/g), arabinose (50-360 mg/g), xylose (approximately 150-270 mg/g), and galactose (approximately 40-140 mg/g), and also contained fucose, rhamnose, and glucose. The ability of the gums to act as stabilizers in whey protein isolate based emulsions varied, with the A. fluccosus gum showing the best emulsion stabilization effect. The emulsion stabilization effect correlated linearly and positively to the methoxylation degree, and galacturonic acid content of the gums, but not to acetyl or fucose content. A particularly high correlation was found between methoxyl level in the soluble gum part and emulsion stabilization. (Ebrahimdazeh, et al., “Mucilage content and its sugar composition in Astragalus species from Iran.” 2000, Pakistan J. of Bot., 32(1): 131-140; Balaghi, et al., “Compositional analysis and rheological characterization of gum tragacanth exudates from six species of Iranian Astragalus.” 2011, Food Hydrocolloids. 25(7):1775-1784; Gavlighi, et al., “Stabilization of emulsions by gum tragacanth (Astragalus spp.) correlates to the galacturonic acid content and methoxylation degree of the gum.” 2013, Food Hydrocolloids. 31(1):5-14).

Described in the following references are various techniques, for example, but not limited to DEAE-Sepharose Fast Flow and Sephacryl S-300 chromatography, ¹H and ¹³C NMR spectroscopy, NOESY and HMBC experiments, which are used to study constituents of Astragalus species:
Kuliev, V. B., and Kasumov, K. N. “Polysaccharides of the gum—Astragalus microcephalus—in the Azerbaijan-SSR USSR.” 1982, Rastit Resur. 18(3): 390-394;
Svechnikova, et al., “Triterpene Glycosides of Astragalus and their Genins XI. Cyclosiversioside G-A Triglycoside from Astagalus sieversianus.” 1983, Chemistry of Natural Compounds 19(3):296-299;
Wang, et al., “Isolation and structural analysis of an acidic polysaccharide from Astragalus membranaceus (Fisch.) Bunge.” 2006, Journal of Integrative Plant Biology, 48(11):1379-1384;
Turska-Szewczuk, “Structural studies of the 0-specific polysaccharide from the lipopolysaccharide of Mesorhizobium huakuii strain S-52, the symbiotic partner of Astragalus sinicus.” 2011, Carbohydrate Research, 346(8):1065-1069;
Kim, et al., “Variation of Astragalosides Contents in Cultivated Astragalus membranaceus.” 2012, Korean J. Medicinal Crop Sci. 20(5):372-380; and
Fu, et al., “Structural features of a polysaccharide from Astragalus membranaceus (Fisch.) Bge. var. mongholicus (Bge.) Hsiao.” 2013, J. Asian Nat. Prod. Res. 15(6):687-692.

In some embodiments, the PGRG1 composition described herein derives from Astragalus species only, for example from Astragalus membranaceus Bge., and does not derive from or include extracts of Dankuei such as polysaccharides from Angelica sinensis Diels, Angelic archangelica or Levisticum officinale Koch.

### The PGRG1 Composition

The isolated PGRG1 composition disclosed and described herein is water soluble and has a weight average molecular weight of 10-50 kDa. It mainly consists of galacturonic acid, rhamnosyl, arabinosyl, galactosyl, glucosyl residues, and has a rhamnose+galacturonic acid total mole % of at least 20% and ratio of rhamnose to galacturonic acid at least 0.06%. A representative sugar composition of this PGRG1 composition is shown in Table 1 below:

TABLE 1
Sugar residue compositions (Mole %)

| | Ara | Rha | GalA | Gal | Glc |
|---|---|---|---|---|---|
| | (Mol %) | (Mol %) | (Mol %) | (Mol %) | (Mol %) |
| PGRG1 | 15.8 | 1.8 | 24.1 | 8.3 | 50.0 |

Rha:GalA ratio (1.8/24.1)=0.0747.

PGRG1 contains about 50% small molecules that were extracted from Astragulas membranaceus.

### Preparation of the PGRG1 Composition

The isolated and purified PGRG1 polysaccharide composition was prepared by first extracting the Astragalus membranaceus with hot water. Typically the clean processed chipped or sectioned dried roots was prepared by trimming the dried roots, cleaning with clean water and a disinfecting solution such as 70% ethanol, cutting into thin slices, and drying under near-aseptic conditions yielding what is referred to as “root chips.” Hot water was typically not less than 80° C., often not less than 90° C., and in some embodiments at about 100° C. In addition, many extraction cycles can be used to yield substantial and/or optimal extraction of the PGRG1 from the roots; typically, at least three cycles for three hours at 100° C. each cycle were performed. All steps following the preparation of the dried comminuted roots were typically conducted under near-aseptic conditions employing clean equipment. The hot water extract was concentrated, such as by evaporation under vacuum at 60-70° C., to a concentration of about 1 L/Kg of “root chips,” and then treated to remove materials not water-soluble, such as by precipitating with a higher concentration of lower alkanol (for example, using 40-80% ethanol, or more particularly 60-70% ethanol), to precipitate the PGRG1 polysaccharide composition. The precipitate was then re-dissolved in water and precipitated again with a lower alkanol concentration, e.g. at 20-40% ethanol, to remove starch. The supernatant was then precipitated with a higher concentration of lower alkanol, e.g. 60-80% ethanol. The precipitate was dried (typically by spray drying, vacuum drying or lyophilizing) to the resultant PGRG1 polysaccharide composition, typically a yellow powder.

### The PGRG1-5K Composition

“PGRG1-5K” is obtained from the polysaccharide fraction of the PGRG1 composition above, after a 5,000 MW cut-off ultrafiltration to remove molecules smaller than 5,000 in MW. PGRG1-5K is approximately 52% of PGRG1 by weight.

Like PGRG1, PGRG1-5K mainly consists of galacturonic acid, rhamnosyl, arabinosyl, galactosyl, glucosyl residues, and has a rhamnose+galacturonic acid total mole % of at least 20% and ratio of rhamnose to galacturonic acid at least 0.06%. A representative sugar composition of this PGRG1 composition is shown in Table 2 below:

TABLE 2
Sugar residue compositions (Mole %)

| | Ara | Rha | GalA | Gal | Glc |
|---|---|---|---|---|---|
| | (Mol %) | (Mol %) | (Mol %) | (Mol %) | (Mol %) |
| PGRG1-5K | 18.2 | 2.0 | 27.3 | 10.1 | 42.0 |

Ratio of Rha:GalA (2.0/27.3)=0.0733.

### Preparation of the Isolated PGRG1-5K Composition

The PGRG1-5K composition was prepared in the same manner as the PGRG1 composition described above except that the PGRG1 precipitate was isolated and ultrafiltered using a 5K MWCO UF system. The retentate from this 5K ultrafiltration was further concentrated, and dried by a spray dryer or via vacuum oven at 60-70° C. after 70% ethanol precipitation of the concentrate, to generate this PGRG1-5K composition.

### Backbone Structure

The backbone structure of PGRG1 and PGRG1-5K compositions as described herein have the following generic structure:

[1,4 and 1,2-Linked RhaGalA]ₘ Interspersed with [1,4-Linked GalAGalA]ₙ Kidney Disease

The kidneys perform several life-sustaining roles including the removal of waste and excess fluid from the blood, the maintenance of salt and minerals in the blood, and the regulation of blood pressure. When the kidneys become damaged, waste products and fluid can build up in the body, causing swelling in the ankles, vomiting, weakness, poor sleep, and shortness of breath. If left untreated, diseased kidneys may eventually stop functioning completely. Loss of kidney function is a serious and potentially fatal. Kidneys also produce renin, an enzyme that helps regulate blood pressure, erythropoietin, which stimulates red blood cell production and an active form of vitamin D, needed for bone health. Kidney disease can be acute or chronic in nature. Acute renal failure can occur as a result of a traumatic injury with blood loss, the sudden reduction of blood flow to the kidneys, damage to the kidneys from shock during a severe infection called sepsis, obstruction of urine flow, such as with an enlarged prostate, damage from certain drugs or toxins, or pregnancy complications.

### Chronic Kidney Disease (CKD)

Chronic kidney disease (CKD) is a progressive loss in renal function over a period of months or years. The symptoms of worsening kidney function are non-specific, and might include feeling generally unwell and experiencing a reduced appetite. Often, chronic kidney disease is diagnosed as a result of screening of people known to be at risk of having a disease or disorder of the kidney, such as those with high blood pressure or diabetes, or those with a relative having chronic kidney disease. CKD may also be identified when it leads to one of its recognized complications, such as cardiovascular disease, anemia or pericarditis. CKD is differentiated from acute kidney disease in that the reduction in kidney function must be present for over 3 months. (National Kidney Foundation. KDOQI Clinical Practice Guidelines for Chronic Kidney Disease: Evaluation, Classification and Stratification. Am. J. Kidney Dis. 39:S1-S000, 2002 (suppl 1)).

G-CSF is reported to accelerate regeneration and prevent apoptosis of renal tubular epithelial cells that leads to reduced renal injury in cisplatin-induced acute renal failure in animal models. (Nishida, et al., 2004, Biochem Biophys Res Commun. 324(1):341-7). A positive effect of hematopoietic cytokines on renal function was observed in cisplatin-induced ARF in mice. Without being bound by theory, it is herein contemplated that, because PGRG1 stimulates G-CSF production in Human peripheral blood mononuclear cells (PBMC), PGRG1-induced elevated G-CSF levels may account (at least in part) for the protective effect of PGRG1 seen in humans with CKD. In CKD, erythropoietin (EPO) is well-supportive as a secondary treatment, as it elevates low red blood cell (RBC) levels, but there are few examples in which EPO truly treats the underlying disease. Thus, a therapeutic effect of PGRG1 and/or the PGRG1-5K composition are contemplated herein.

CKD is identified by a blood test for creatinine. Higher levels of creatinine indicate a lower glomerular filtration rate and as a result a decreased capability of the kidneys to excrete waste products. Creatinine levels may be normal in the early stages of CKD, and the condition is discovered if urinalysis (testing of a urine sample) shows that the kidney is allowing the loss of protein or red blood cells into the urine. To fully investigate the underlying cause of kidney damage, various forms of medical imaging, blood tests and often renal biopsy (removing a small sample of kidney tissue) are employed to find out if there is a reversible cause for the kidney malfunction. (National Kidney Foundation. KDOQI Clinical Practice Guidelines for Chronic Kidney Disease: Evaluation, Classification and Stratification. Am. J. Kidney Dis. 39:S1-S000, 2002 (suppl 1). Recent medical guidelines classify the severity of chronic kidney disease in five stages, with stage 1 being the mildest and usually causing few symptoms and stage 5 being a severe illness with poor life expectancy if untreated. Stage 5 CKD is often called end stage renal disease (ESRD) or end stage renal failure (ESRF) and is synonymous with the now outdated terms chronic kidney failure (CKF) or chronic renal failure (CRF).

### Renal Function

Renal function, in nephrology, is an indication of the state of the kidney and its role in renal physiology. Glomerular filtration rate (GFR) describes the flow rate of filtered fluid through the kidney. Creatinine clearance rate (C_{Cr} or CrCl) is the volume of blood plasma that is cleared of creatinine per unit time and is a useful measure for approximating the GFR. Creatinine clearance exceeds GFR due to creatinine secretion.

The GFR is typically recorded in units of volume per time, e.g., milliliters per minute mL/min. Compare to filtration fraction.

$$GFR = \frac{Urine Concentration \times Urine Flow}{Plasma Concentration}$$

In clinical practice, however, creatinine clearance or estimates of creatinine clearance based on the serum creatinine level are used to measure GFR. A commonly used surrogate marker for estimate of creatinine clearance is the Cockcroft-Gault (CG) formula, which in turn estimates GFR in ml/min: It is named after the scientists who first published the formula, and it employs serum creatinine measurements and a patient's weight to predict the creatinine clearance. The formula, as originally published, is:

$${eC}_{Cr} = \frac{( 140 - Age ) \times Mass ( in kilograms ) \times [ 0.85 if Female ]}{72 \times Serum Creatinine ( in mg \text{/} dL )}$$

One estimation tool to calculate GFR is the Mayo Quadratic formula. This formula was developed by Rule in an attempt to better estimate GFR in patients with preserved kidney function. It is well recognized that the MDRD formula tends to underestimate GFR in patients with preserved kidney function.

The equation is:

eGFR=exp(1.911+5.249/Serum Creatinine−2.114/Serum Creatinine²−0.00686×Age−[0.205 if Female])

### Leukopenia, Anemia and Thrombocytopenia

Blood is a multifaceted body fluid and the medium through which essential nutrients are delivered to tissues throughout the body. On average, the adult human body contains more than 5 liters of blood. Blood flows freely through the veins and arteries because it is over half liquid plasma; the remainder of blood volume consists mostly of solid cells and cell fragments, which are suspended in the plasma. The three major blood disorders are anemia, leukopenia, and thrombocytopenia.

### Leukopenia

Leukopenia is a decrease in the number of white blood cells (WBCs) found in the blood, which places individuals at increased risk of infection. Neutropenia, a subtype of leukopenia, refers to a decrease in the number of circulating neutrophil granulocytes, the most abundant white blood cells. The terms leukopenia and neutropenia may occasionally be used interchangeably, as the neutrophil count is the most important indicator of infection risk. Low white cell count may be due to acute viral infections, such as with a cold or influenza. It can be associated with chemotherapy, radiation therapy, myelofibrosis and aplastic anemia. HIV and AIDS are also a threat to white cells. Other causes of low white blood cell count include systemic lupus erythematosus, Hodgkin's lymphoma, some types of cancer, typhoid, malaria, tuberculosis, dengue, rickettsial infections, enlargement of the spleen, folate deficiencies, psittacosis and sepsis. Many other causes exist, such as deficiency in certain minerals, such as copper and zinc. Drugs that can impact the number and function of white blood cells include clozapine, bupropion, Minocycline, valproic acid, lamotrigine, and metronidazole. Other drugs that depress WBC count are immunosuppressants, such as sirolimus, mycophenolate mofetil, tacrolimus, cyclosporine and TNF inhibitors. Interferons used to treat multiple sclerosis, such as Rebif, Avonex, and Betaseron, can also cause leukopenia. Chemotherapy targets cells that grow rapidly, such as tumors, but can also impact white blood cells, because they are characterized by bone marrow as rapid growing. A common side effect of cancer treatment is neutropenia, the lowering of neutrophils (a specific type of white blood cell).

### Anemia

Anemia is a decrease in number of red blood cells (RBCs). Anemia in its broadest sense is also less than the normal quantity or quality of hemoglobin in the blood. It includes a decreased oxygen-binding ability of each hemoglobin molecule due to deformity or a lack in numerical development. Since all human cells depend on oxygen for survival, varying degrees of anemia can have a wide range of clinical consequences. Anemia is the most common blood disorder, goes undetermined in many people, and symptoms can be minor. Most commonly, people report feelings fatigue or shortness of breath on exertion. In very severe anemia, the body may increase cardiac output. Anemia is typically diagnosed on a complete blood count that reports the number of RBCs and the hemoglobin level. There are more than 400 types of anemia, which are divided into three causes: blood loss, decreased RBC production, or destruction of RBCs. RBCs can be lost through bleeding, which can occur slowly over a long period of time, and can often go undetected. This kind of chronic bleeding commonly results from gastrointestinal conditions such as ulcers, hemorrhoids, gastritis (inflammation of the stomach), and cancer, use of nonsteroidal anti-inflammatory drugs (NSAIDS) such as aspirin or ibuprofen, or menstruation and childbirth in women, especially if menstrual bleeding is excessive and if there are multiple pregnancies. With anemia caused by decreased or faulty RBC production, the body may produce too few blood cells or the blood cells may not function correctly. In either case, anemia can result. RBCs may be faulty or decreased due to abnormal RBCs or a lack of minerals and vitamins needed for RBCs to work properly. Conditions associated with these causes of anemia include sickle cell anemia, iron-deficiency anemia, vitamin deficiency, bone marrow and stem cell problems.

### Thrombocytopenia

Thrombocytopenia is the medical term for a low blood platelet count. Platelets (thrombocytes) are colorless blood cells that play an important role in blood clotting. Platelets stop blood loss by clumping and forming plugs in blood vessel holes. Thrombocytopenia may be mild and cause few signs or symptoms. In rare cases, the number of platelets may be so low that dangerous internal bleeding can occur. Thrombocytopenia usually improves when the underlying cause is treated. Sometimes medications, surgery or a blood transfusion can help treat chronic thrombocytopenia. Low platelet counts, thrombocytopenia, can be caused by a variety of reasons and can be divided into decreased platelet production, increased platelet destruction or consumption, or increased splenic sequestration. Decreased platelet production is usually related to a bone marrow problem. In some of these conditions, red blood cell and white blood cell productions may also be affected. Viral infections affecting the marrow for example: parvovirus, rubella, mumps, varicella (chickenpox), hepatitis C, Epstein-Barr virus, and HIV. Aplastic anemia is a general term used when the bone marrow fails to produce any blood cells. This can be caused by some viral infections (parvovirus or HIV), medications (gold, chloramphenicol, Dilantin, valproate (Depakote), or radiation. Chemotherapy drugs frequently cause thrombocytopenia. Some other drugs can suppress platelet production, such as thiazide diuretics. Cancers of the bone marrow and blood or cancers of the lymph nodes can cause various degrees of thrombocytopenia. Long term alcohol can cause direct toxicity of the bone marrow. Deficiency of vitamin B12 and folic acid can result in low platelet production by the bone marrow. Increased platelet destruction or consumption can be seen a number of medical conditions. Many medications can cause low platelet count by causing immunologic reaction against platelets, and include sulfonamide antibiotics, carbamazepine, digoxin, quinine, quinidine, acetaminophen, rifampin, and heparin. Idiopathic thrombocytopenic purpura (ITP) is a condition where the immune system attacks platelets. Some rheumatologic condition, such as systemic lupus erythematosus (SLE) or other autoimmune conditions, can cause platelet destruction including transfusion of blood products and organ transplantation, thrombotic thrombocytopenic purpura (TTP) and hemolytic uremic syndrome (HUS), HELLP syndrome, and disseminated intravascular coagulopathy (DIC). Splenic sequestration can also lead to low platelet counts as a result of enlargement or change in function of the spleen for a variety of reasons. Common causes of thrombocytopenia due to splenic enlargement may include advanced liver disease with portal hypertension (cirrhosis, for example, from chronic hepatitis B or C) and blood cancers (leukemias or lymphomas).

### Immune Disorders

An immune disorder is a dysfunction of the immune system. These disorders can be characterized by the component(s) of the immune system affected, by whether the immune system is overactive or underactive, or by whether the condition is congenital or acquired. According to the International Union of Immunological Societies, more than 150 primary immunodeficiency diseases (PIDs) have been characterized. However, the number of acquired immunodeficiencies exceeds the number of PIDs. It has been suggested that most people have at least one primary immunodeficiency (Casanova and Abel. 2007, “Primary immunodeficiencies: a field in its infancy.” Science 317(5838):617-9). However, due to redundancies in the immune system, many of these go undetected.

Autoimmune diseases arise from an abnormal immune response of the body against substances and tissues normally present in the body. This may be restricted to certain organs (e.g. in autoimmune thyroiditis) or involve a particular tissue in different places (e.g. Goodpasture's disease which may affect the basement membrane in both the lung and the kidney). The treatment of autoimmune diseases is typically with immunosuppression—medication that decreases the immune response. A large number of autoimmune diseases are recognized. A major understanding of the underlying pathophysiology of autoimmune diseases has been the application of genome wide association scans that have identified a striking degree of genetic sharing among the autoimmune diseases. A partial list of some autoimmune disorders include lupus, scleroderma, certain types of hemolytic anemia, vasculitis, type one diabetes, Graves disease, rheumatoid arthritis, multiple sclerosis, Goodpasture's syndrome, Pernicious anemia, some types of myopathy and late Lyme disease.

There are a large number of immunodeficiency syndromes that present clinical and laboratory characteristics of autoimmunity. The decreased ability of the immune system to clear infections may be responsible for causing autoimmunity through perpetual immune system activation. Examples include common variable immunodeficiency (CVID) where multiple autoimmune diseases are seen, e.g. inflammatory bowel disease, autoimmune thrombocytopenia and autoimmune thyroid disease, and familial hemophagocytic lymphohistiocytosis (FHL), an autosomal recessive primary immunodeficiency, where pancytopenia, rashes, lymphadenopathy and hepatosplenomegaly are commonly seen.

### Chronic Kidney Disease (CKD) and Utility

The utility of this disclosure is to reduce creatinine levels in animals which is indicative of slowing the CKD process. There is no specific treatment unequivocally shown to slow the worsening of chronic kidney disease. If there is an underlying cause to CKD, such as vasculitis, this may be treated directly to slow the damage. In more advanced stages, treatments may be required for anemia and bone disease. Severe CKD requires renal replacement therapy, which may involve a form of dialysis, but ideally constitutes a kidney transplant. (National Kidney Foundation. KDOQI Clinical Practice Guidelines for Chronic Kidney Disease: Evaluation, Classification and Stratification. Am J Kidney Dis 39:S1-S000, 2002 (suppl 1).

### Leukopenia, Anemia and Thrombocytopenia, and Utility

The utility of this disclosure is to increase WBCs, RBCs and platelets in animals with leukopenia, anemia and thrombocytopenia, respectively. Conventional treatment of these blood disorders is often hindered by significant side effects, and in some severe cases, patients must undergo invasive procedures or take medications for the rest of their lives. However, emerging therapeutic technologies, such as gene therapy, may improve the outlook for anemia in the near future. Moreover, some blood disorders may be caused by conditions that are easily treatable, but often underappreciated. For example, in men, low testosterone can cause anemia, and testosterone replacement therapy has been shown to promote healthy red blood cell production in this population.

### Autoimmune Diseases and Utility

The utility of this disclosure is to modulate the immune system that is overactive or underactive. The goals of treatment are to reduce symptoms, control the autoimmune process and to maintain the body's ability to fight disease. Which treatments are used depends on the specific disease and symptoms. Some patients may need supplements to replace a hormone or vitamin that the body is lacking. Examples include thyroid supplements, vitamins such as B12, or insulin injections. If the autoimmune disorder affects the blood, you may need blood transfusions. Medicines prescribed to control or reduce the immune system's response include corticosteroids (such as prednisone) and nonsteroid drugs such as azathioprine, cyclophosphamide, mycophenolate, sirolimus, or tacrolimus.

### Dietary Supplement Health and Education Act of 1994 (“DSHEA”)

The Dietary Supplement Health And Education Act of 1994 (“DSHEA”), is a 1994 statute of United States Federal legislation which defines and regulates dietary supplements. DSHEA defines the term “dietary supplement” to mean a product (other than tobacco) intended to supplement the diet that bears or contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any of the aforementioned ingredients. Furthermore, a dietary supplement must be labeled as a dietary supplement and be intended for ingestion and must not be represented for use as conventional food or as a sole item of a meal or of the diet. In addition, a dietary supplement cannot be approved or authorized for investigation as a new drug, antibiotic, or biologic, unless it was marketed as a food or a dietary supplement before such approval or authorization. Under DSHEA, dietary supplements are deemed to be food, except for purposes of the drug definition

### Pharmaceutical Formulations and Administration

In general, the purified PGRG1 composition of the first aspect of this disclosure and the PGRG1-5K composition of the second aspect of this disclosure will be administered in therapeutically effective amounts by oral administration, either singly or in conjunction with of at least one other therapeutic agent capable of inhibiting Chronic kidney disease. A therapeutically effective amount may vary widely depending on the disease, its severity, the age and relative health of the animal being treated, and other factors. A person of ordinary skill in the art will be able without undue experimentation, having regard to that skill and this disclosure, to determine a therapeutically effective amount of the compositions of this disclosure for a given disease.

In general, the purified PGRG1 composition of the first aspect of this disclosure and the PGRG1-5K composition of the second aspect of this disclosure will be administered alone or as pharmaceutical formulations, which in some embodiments may be administered orally. In pharmaceutical formulations, the composition comprising the purified PGRG1 composition of the first aspect of this disclosure or the PGRG1-5K composition of the second aspect of this disclosure may occur in combination with another active agent and/or an oral excipient. Suitable oral excipients are well known to persons of ordinary skill in the art.

Typically, when administered as an anti-nephritis agent, the purified PGRG1 composition and the PGRG1-5K composition of the present disclosure will be administered orally. When formulated, the amount of a compound in the composition of the present disclosure may vary widely depending on the type of composition, size of a unit dosage, kind of excipients, and other factors well known to those of ordinary skill in the art. In general, if administered alone the amount of compound may vary from 400 mg to 4 grams.

The purified PGRG1 composition and the PGRG1-5K composition of the present disclosure may optionally be administered in conjunction with of at least one other therapeutic agent capable of ameliorating nephritis selected from, for example, an angiotensin converting enzyme inhibitor (ACEI), such as, linsinopril.

### EXAMPLES

The following non-limiting examples highlight the features of the invention(s). However, the present disclosure shall in no way be considered to be limited to the particular embodiments described below.

All commercially available materials were used as received. All isolated materials were characterized by protein content, sugar content, sugar residue analysis, molecular weight distribution, and sugar linkage analysis.

### Example 1

### Preparation of the PGRG1 Composition

Step A. Root Chip Processing

Dried Astragalus membranaceus raw materials (roots) (220 kg) was processed into root chips by sterile washing with ultrafiltered water, removing the contaminated parts, soaking in 70% ethanol overnight, cutting into chips with a thickness of 0.3-0.5 mm, and near sterile oven drying at 60-70° C. Approximately 200 kg of dried chips are obtained with a loss on drying of <10%.

Step B. Polysaccharide Composition Extraction and Purification

Dried root chips (200 kg) are extracted with ultra-filtered water at about 100° C. for 3 times, each time for 3 hours. The water added in the first extraction was 2,000 L, in the second was 1,600 L, and in the third was 1,200 L. A total combined water extract of about 4,500 L was obtained, that was concentrated using a vacuum concentrator system at 60-80 C, to obtain a concentrate at a volume about 200 L. The 200 L of extract was precipitated with 70% ethanol to remove water insoluble small molecules. The precipitate obtained was then suspended in water and precipitated with 30-35% ethanol to remove lower water soluble polysaccharides, proteins and other larger molecules. The supernatant was re-precipitated at 70% ethanol to recover the highly water soluble polysaccharides or PGRG1. The precipitate was dissolved in water and spray-dried to obtain PGRG1 (7 kg or 3.5% of dried drink chips). The resulting PGRG1 composition is a yellow powder, water soluble, pH 5-6, loss on drying <6%.

Flow chart 1

Dried Astragalus roots (220 kg)
Drink chip processing

Dried root chips (200 kg)
extracted three times with water at 100° C., (first extraction 2,000 L water, second extraction 1,600 L water, third extraction 1,200 L water).

Water extract (4,500 L)
Concentrated at 60-80 C, to remove water

Concentrated extract (200 L)
70% ethanol precipitation—1, to remove water-insoluble molecules
30% ethanol precipitation to remove water insoluble polysaccharides
70% ethanol precipitation—2, to precipitate RGI polysaccharides
Spray dry, to recover dried RGi polysaccharides

PGRG1 yield (7 kg, 3.5% from root chips)

### Example 2

### Preparation of the PGRG1-5K Composition

Step A. Root Chip Processing—See Example 1, Step A.

Step B. Polysaccharide Composition Extraction and Purification

Dried root chips (200 kg) are extracted with ultra-filtered water at about 100° C. for 3 times, each time for 3 hours. The water added in the first extraction was 2,000 L, in the second was 1,600 L, and in the third was 1,200 L. A total combined water extract of about 4,500 L was obtained, that was concentrated using a vacuum concentrator system at 60-80 C, to obtain a concentrate at a volume about 200 L. The 200 L of extract was precipitated with 70% ethanol to remove water insoluble small molecules. The precipitate obtained was then suspended in water and precipitated with 30-35% ethanol to remove lower water soluble polysaccharides, proteins and other larger molecules. The supernatant was re-precipitated at 70% ethanol to recover the highly water soluble polysaccharides or PGRG1. The precipitate was dissolved in water and spray dried to obtain PGRG1 (7 kg or 3.5% of dried drink chips).

Step C. Polysaccharide Composition Isolation

To obtain the PGRG1-5K compositions, the 3.5 kg PGRG1 was isolated and ultra-filtered using a UF system with a membrane of MWCF 5,000 Daltons. The ultra-filtration was circulated for 5 cycles, each cycle concentrated down to about 50% in volume, to remove small molecules. The concentrate was precipitated at 70% ethanol, vacuum oven dried or spray dryer dried after suspension in water, to obtain PGRG1-5K (1.82 kg). This further-purified PGRG1 polysaccharide compositions (PGRG1-5K) is a light-yellow powder, water soluble, pH 5-6, loss on drying <6%.

Flow chart 2

Dried Astragalus roots (220 kg)
Drink chip processing

Dried drink chips (200 kg)
extracted three times with water at 100° C., (first extraction 2,000 L water, second extraction 1,600 L water, third extraction 1,200 L water).

Water extract (4,500 L)
Concentrated at 60-80 C, to remove water

Concentrated extract (200 L)
70% ethanol precipitation—1, to remove water-insoluble molecules
30% ethanol precipitation to remove water insoluble polysaccharides
70% ethanol precipitation—2, to precipitate RGI polysaccharides
Spray dry, to recover dried RGi polysaccharides

RGI compositions (7 kg, 3.5% of drink chips)
Ultrafiltration at 5,000 MWCF, 5 cycles at 50% volume of each cycle, to remove water soluble small molecules
70% ethanol precipitation
Vacuum oven drying, or spray drying

PGRG1-5K composition (1.82 kg)

### Analysis of the Isolated, Purified PGRG1 Composition:

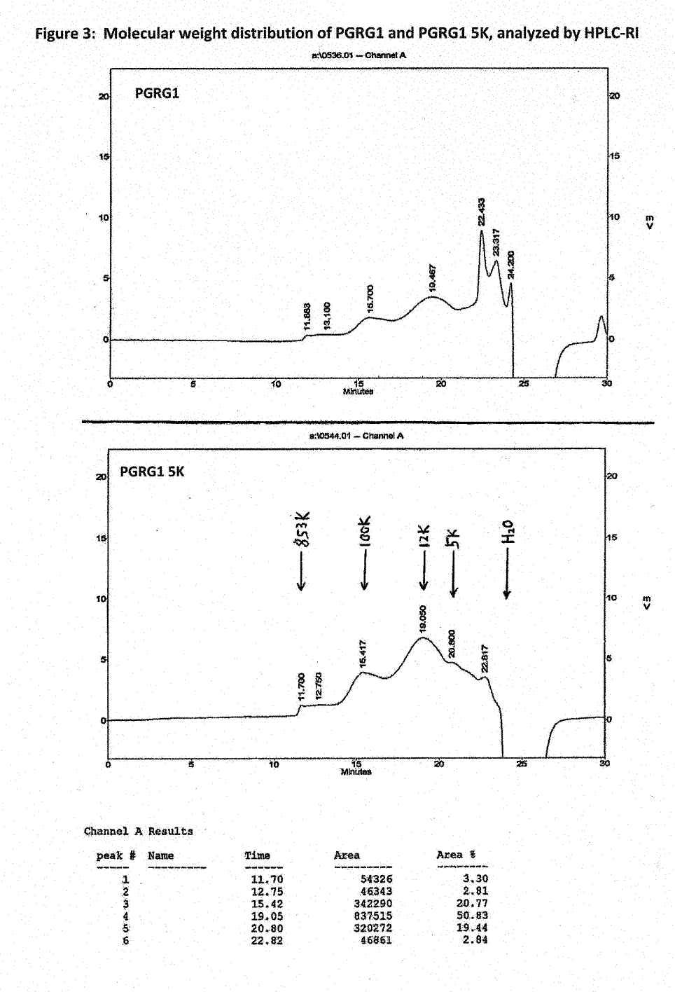

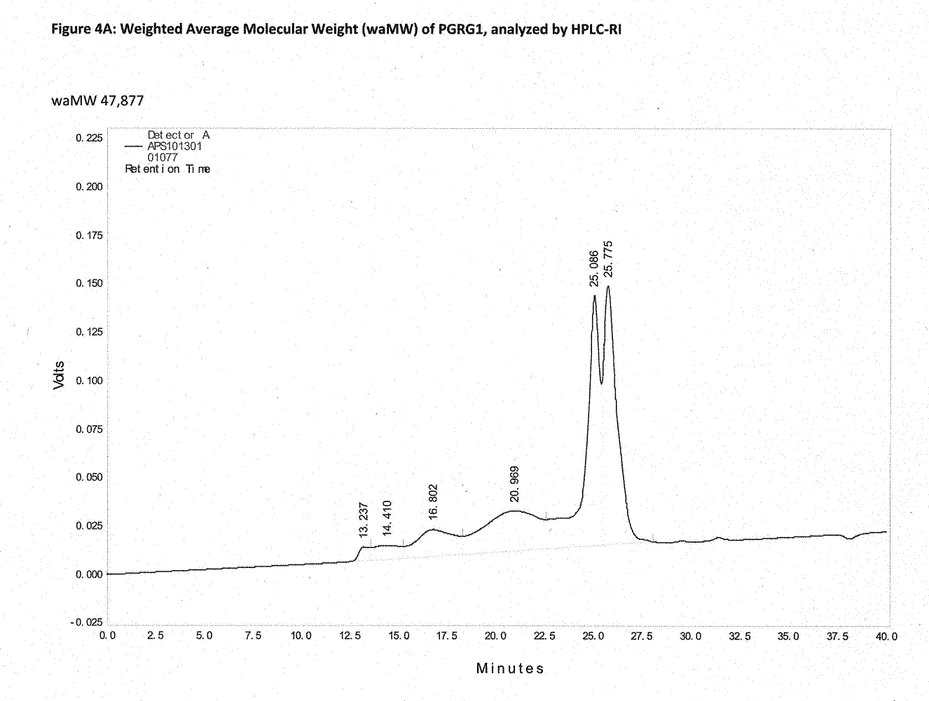

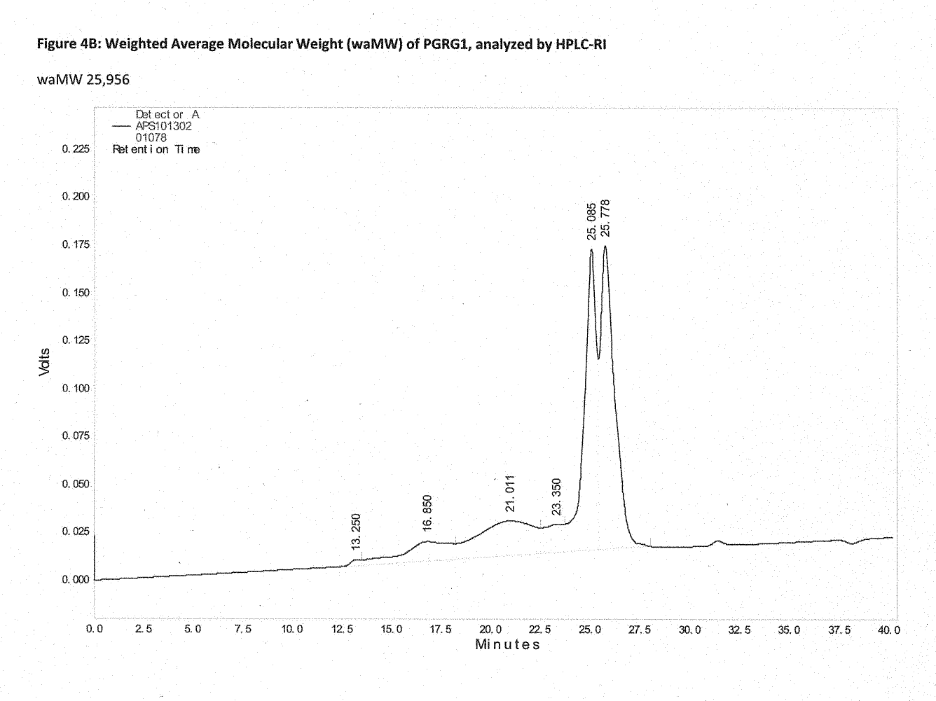

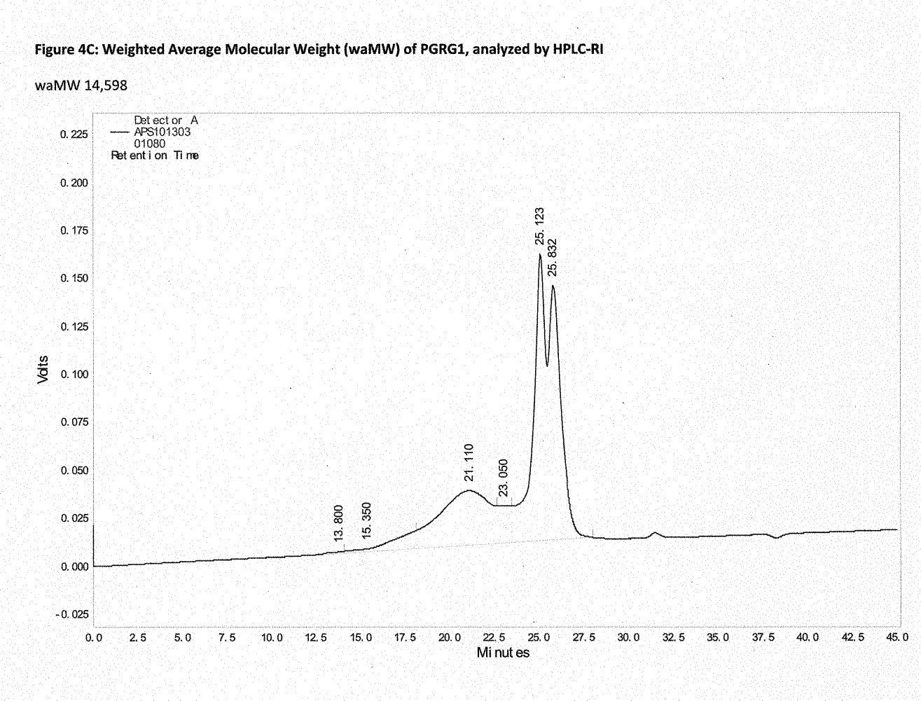

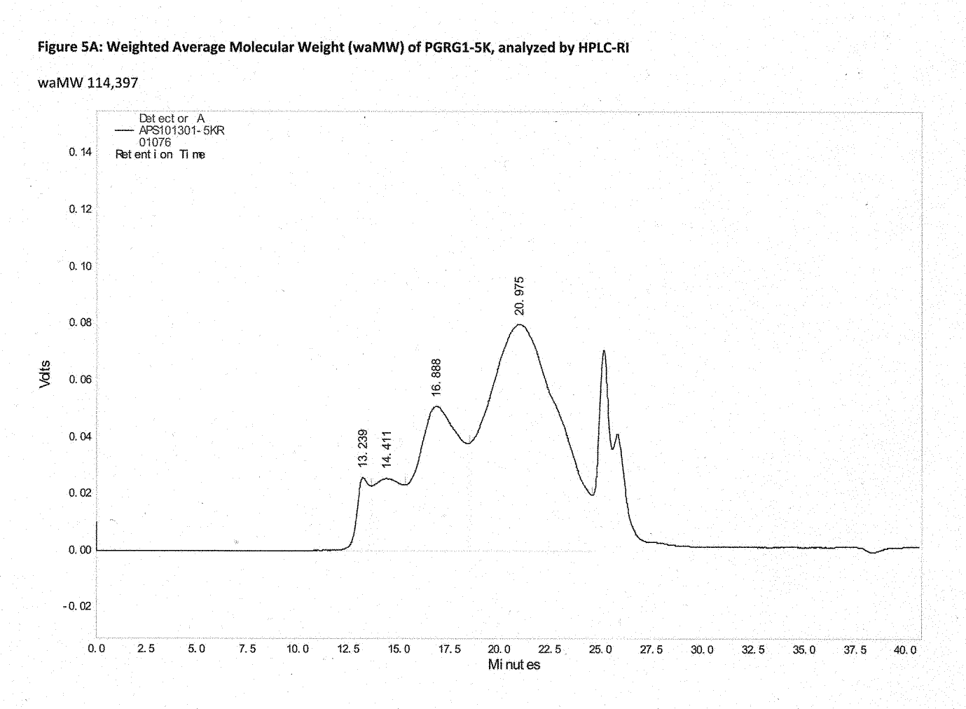

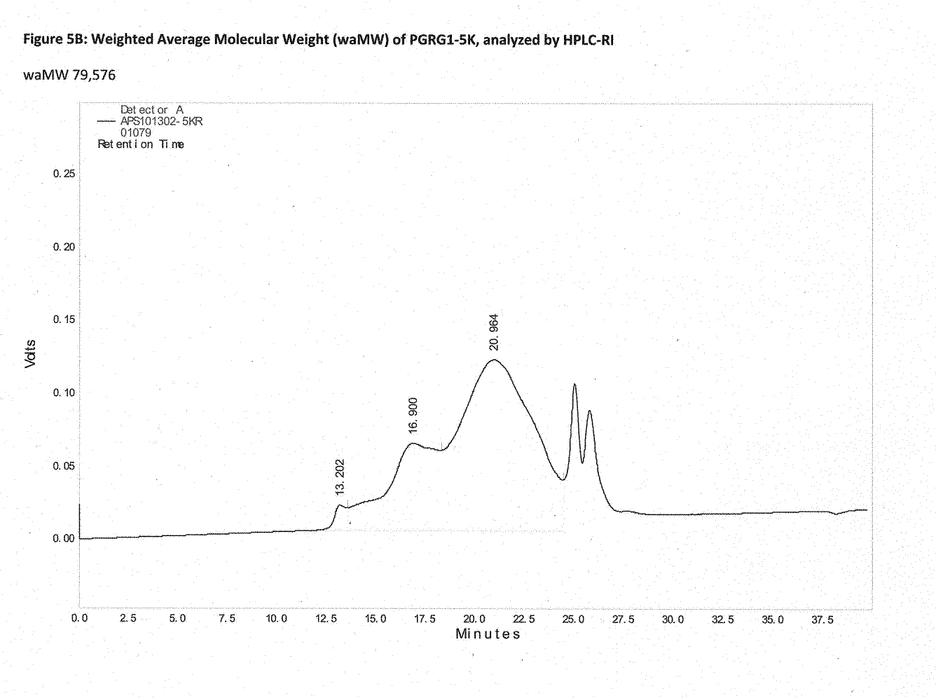

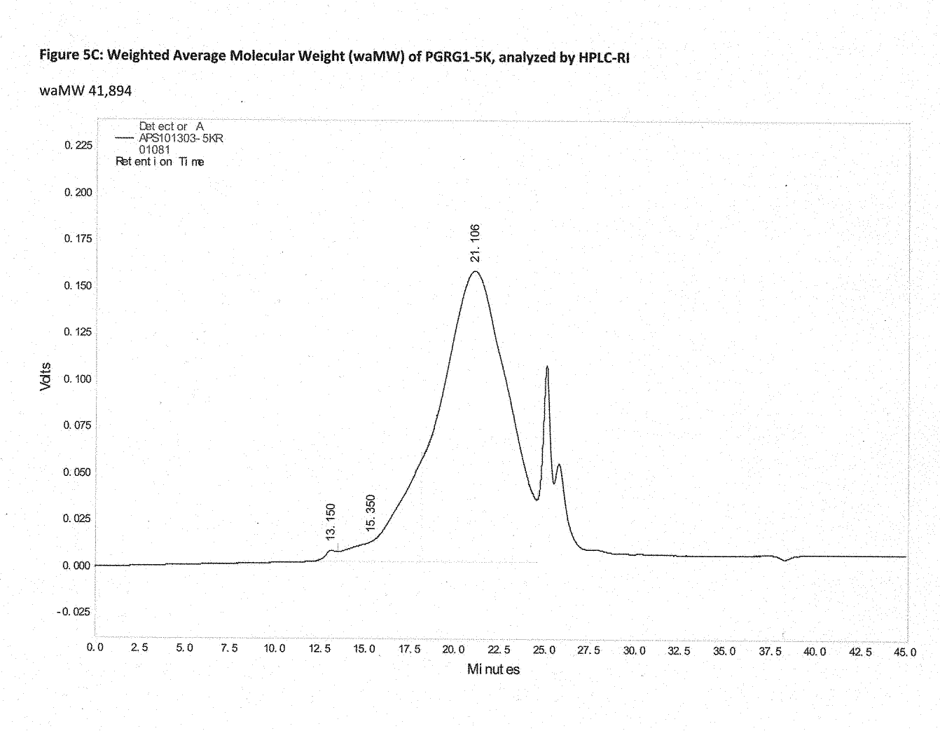

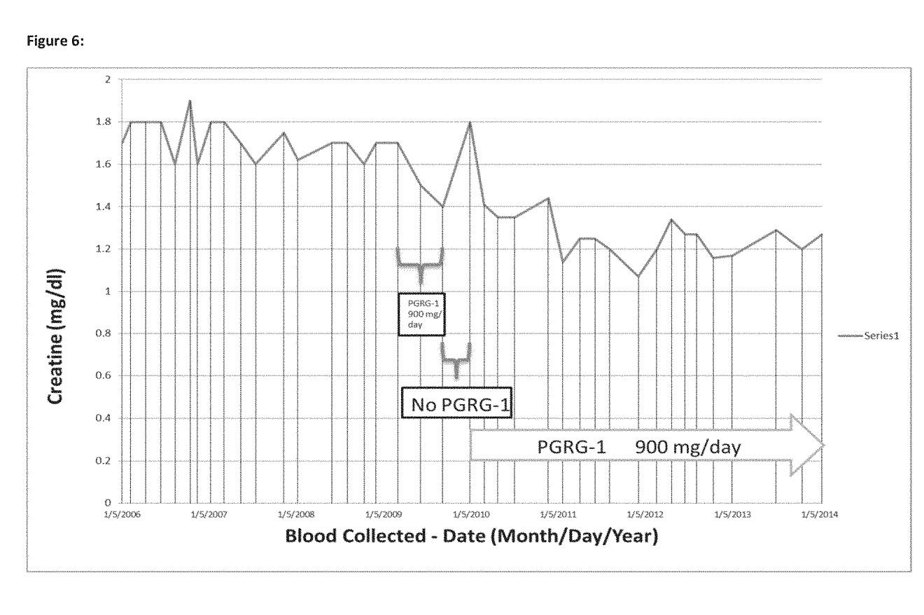

PGRG1, a water soluble polysaccharide composition, was analyzed for protein content by Bio-Rad Method providing a 2.28 wt % result, sugar content was analyzed by Ph-H₂SO₄ method giving a 42.2 wt % result, a sugar composition by TMS derivatives-GC method results are summarized in Table 3 below, a MW distribution and determination by GPC-HPLC-RID in FIG. 3, and the glycosyl-linkage composition by methylation-GC-MS is discussed below.

TABLE 3
PGRG1 Glycosyl-residue compositions, analyzed TMS
derivatives/GC method:

| | Ara | Rha | GalA | Gal | Glc |
|---|---|---|---|---|---|
| | (Mol %) | (Mol %) | (Mol %) | (Mol %) | (Mol %) |
| PGRG1 | 15.8 | 1.8 | 24.1 | 8.3 | 50.0 |

PGRG1 protein content is 2.28 wt %, and its sugar content is 42.2 wt %.

PGRG1 sugar composition is shown in Table 3. At least five sugar residues were identified therein, which were arabinosyl (Ara), rhamnosyl (Rha), galacturonic acid (GalA), galactosyl (Gal), and glucosyl (Glc) residues.

PGRG1 molecular weight (MW) or weight averaged molecular weight (waMW) was determined by GPC/HPLC as shown in FIG. 3. Polysaccharides are larger than 10 kDa and range from 10 kDa to 50 kDa. Employing pullulan standards, these polysaccharides can be divided into two MW ranges: one type of polysaccharide is centered around 100 kDa, and the other type is centered around 12 kDa. In summary, polysaccharides in PGRG1 account for approximately 50% based on the molecular weight distribution, and PGRG1 has a weight averaged molecular weight (waMW) from 10 kDa to 50 kDa.

PGRG1's glycosyl-linkages of the polysaccharides were determined by their derivatives of partially methylated alditol acetates. The carboxyl groups of uronic acid residues were reduced by Super Deuteride followed by acetylation. A GC-MS system was used for analysis. The major glycosyl-linkages are as follows: Araf 5-, 2,5-, and 3,5-linked; Galp: 6-, and 3,6-linked; Glc: p 4-, 4,6-linked; Glcp: 4-, and 4,6-linked; Rhap: 2-, and 2,4-linked; GalAp: 4-, and 2,4-linked, in addition to terminal linkages.

### Analysis of PGRG1-5K:

PGRG1-5K, also a water soluble polysaccharide composition, has been analyzed for protein Content by Bio-Rad Method 2.97 wt % analyzed, sugar content by Ph-H₂SO₄ method 44.9 wt % analyzed, a sugar composition by TMS derivatives—GC method as shown in Table 4, a MW distribution and determination by GPC-HPLC-RID in the lower panel of FIG. 3, and glycosyl-linkage composition by methylation-GC-MS.

Its protein content is 2.97 wt %, and its sugar content is 44.9 wt %.

For its sugar compositions as shown in Table 4 below, five sugar residues are identified as well, which are arabinosyl, rhamnosyl, galacturonic acid, galactosyl, and glucosyl residues.

PGRG1-5K molecular weight or average-weighted molecular weight was determined by GPC-HPLC to be 50-120 k as shown in FIG. 3. The higher molecular weight (>5K) curve is the same as for PGRG1, because PGRG1-5K is the polysaccharide part of PGRG1 after the small molecules are removed. This is also true for its glycosyl-linkage compositions determined by the same methylation-GC/MS method.

TABLE 4
Glycosyl-residue compositions of PGRG1-5K, analyzed
TMS derivatives—GC method:

| | Ara | Rha | GalA | Gal | Glc |
|---|---|---|---|---|---|
| | (Mol %) | (Mol %) | (Mol %) | (Mol %) | (Mol %) |
| PGRG1-5K | 18.2 | 2.0 | 27.3 | 10.1 | 42.0 |

### Structures of PGRG1 and PGRG1-5K Polysaccharides:

Based on the analysis of PGRG1 and PGRG1-5K, in particular glycosyl residue compositions, glycosyl-linkage compositions, and MW distributions, the polysaccharides in PGRG1 and in PGRG1-5K consist mainly of the polysaccharides polygalacturonan rhamnogalacturonans and glucans. The polygalacturonan rhamnogalacturonans present in PGRG1 and in PGRG1-5K is composed of a backbone consisting of [1,4 and 1,2-linked RhaGalA]ₘ interspersed with [1,4-linked GalAGalA]ₙ and side-chains of various sizes that made largely out of neutral sugar residues consisting of Gal, Ara, and Glc linked to the 4 position of Rha. The glucans are α-1,4 (1,6)-glucans with a backbone of [1,4 linked Glc]ₙ and side chains made of Glc linked to the backbone Glc mostly through the 6 position.

### Example 3

### CKD Clinical Data

A patient presented with high blood pressure and blood analysis indicated higher than normal creatinine levels (>1.1 mg/dl). After stabilizing the patient's blood pressure within normal ranges employing ACE inhibitor, treatment with PGRG1 was initiated (see Example 6).

Examination of the results indicates upon initial treatment (oral 900 mg daily) with PGRG1 there was a subsequent drop in creatinine levels. Discontinuation of PGRG1 treatment resulted in higher levels of creatinine. Re-initiation of treatment resulted in lowering of creatinine to high-normal range (<1.4 mg/dl).

Another way to look at this data is to use calculated gel filtration rate (cGFR).

### Example 4

### In Vitro Assays

In vitro assays can be used to evaluate the ability of PGRG1 to enhance immune and hematopoietic functions. These assays were can also be used to provide a convenient assay system for the development of a purified, active PGRG1 fraction having standardized pharmacologic activity. PGRG1 extracts shown to be active in the in vitro assays can then be checked in appropriate in vivo models to evaluate its efficacy (see accompanying in vivo activity). Mouse spleen cell proliferation and cytokine production by human peripheral blood mononuclear cells (PBMC) can be used to show whether PGRG1 extracts have potential immunostimulatory and hematopoietic activity. The cytokines measured were IL-6, and G-CSF, since these cytokines are involved in hematopoiesis and immune functions. PBMC are easy to prepare as well as relevant for studying immune and hematopoietic functions.

### C3H/HeJ Mouse Spleen Cell Proliferation

Spleen cells from C3H/HeJ female mice, 6 to 8 weeks of age (Jackson Laboratories, Bar Harbor, Me.), were prepared as follows. The spleens were removed from the mice and placed into 10 ml of cold HBSS in a sterile petri dish. The spleen was cut in half and gently pressed between the frosted ends of 2 sterile microslides. The cell suspension was then filtered through sterile nylon mesh (Nytex, Tetco #HD-3-85) into a 15 ml conical polypropylene centrifuge tube and centrifuged at 200×g for 10 minutes in a Beckman GPR tabletop centrifuge (GH-3.7 Rotor). Following an additional wash in HBSS, the spleen cells were resuspended as 2.5×106 cells/ml in RPMI 1640 medium (Gibco) containing 50 μM 2-mercaptoethanol, 2 mM glutamine, 1 mM sodium pyruvate, non-essential amino acids, 100 U/ml penicillin, 100 μg/ml streptomycin and 10% heat-inactivated fetal bovine serum (Sigma, #F1442).

Triplicate aliquots containing 100 μl of cells and 100 μl of test samples at various concentrations were incubated in round bottom 96 well plates (Costar #3799) at 37° C. in a 5% CO2 incubator for three days. Six wells containing 100 μl of cells and 100 μl of medium served as controls. Fifty microliters of medium containing 1 μCi of [3H]thymidine (Amersham, 49 Ci/mmol) was added to each wells prior to the last 18 hours of incubation. Cells were then harvested onto filtermats (glass fiber filter, Wallac) using a cell harvester per manufacturer's instruction (Harvester 96, Tomtec Inc.). Samples were processed and counted in a liquid scintillation counter (1205 Betaplate, Pharmacia LKB Biotechnology, Inc.) and the results were reported as counts per minute (cpm) per 2.5×105 cells.

Results are expressed as mean of triplicate determinations +/−standard deviation (SD). Control values represent the mean of six separate determinations. Values are also expressed as the proliferation index (PI), where mean number of counts per minute (cpm) in sample cultures was divided by mean cpm in control cultures. Typical control values ranged from 2000 to 6000 cpm.

In the C3H/HeJ proliferation assay, results are expressed as mean of triplicate determinations +/−standard deviation (SD). Control values represent the mean of six separate determinations. Significance was determined using the unpaired student t-test.

### Cytokine Production by Activated Human PBMC

Human peripheral blood mononuclear cells (PBMC) were prepared using an established method. Human blood buffy coat samples, approximately 25 ml/donor, were obtained from the Stanford University Medical Center Blood Bank. Using sterile techniques, the buffy coat samples were gently resuspended in a total volume of 100 ml with the addition of calcium and magnesium free Hank's balanced salt solution (HBSS, Gibco) at room temperature. A volume of 25 ml of the cell suspension was then layered onto 15 ml of Ficoll-Paque (Pharmacia LKB Biotechnology, Inc.) in a 50 ml conical centrifuge tube. Tubes were centrifuged in a Beckman GPR tabletop centrifuge (GH-3.7 Rotor) at 400×g for 30 minutes at 15° C.

Following centrifugation, the PBMC suspensions at the interfaces were transferred to new 50 ml tubes, resuspended in a total volume of 45 ml HBSS, and centrifuged at 354×g for 10 minutes at 15° C.

Supernatants were discarded, cell pellets were resuspended to a total of 45 ml with HBSS, and centrifuged again at 265×g for 10 minutes at 15° C. The cell pellets were resuspended in 10 ml of X-Vivo tissue culture medium (Bio Whittaker, MD) and counted using a hemocytometer. Polystyrene tubes (Falcon #2057, Becton Dickinson) and PBMC from 2 different donors were used in the following set up. PBMC suspensions were diluted to 4×10 6/ml; 1 ml was incubated in the presence of 0.5 ml of phytohemagglutinin P (PHA-P, Pharmacia 27-3707-01) at a final concentration of 3 μg/ml together with 0.5 ml of test samples at various concentrations. Total volume per tube was 2 ml. Another aliquot of cells treated with PHA alone served as control. After 24 hours incubation at 37° C. in an incubator with 7% CO₂, the tubes were centrifuged in a Beckman GPR tabletop centrifuge (GH-3.7 Rotor) at 1600×g for 10 minutes at 15° C., supernatants were collected and stored at −70° C. prior to assay. Cell pellets were stained with Trypan blue to assess viability as described below.

Cytokine measurements were carried out using commercially available ELISA assay kits for human cytokines IL-1β, IL-6, TNF-α, GM-CSF and G-CSF (R&D Systems, MN) and human IFN-γ (Endogen, MA) in accordance with the manufacturer's protocols. Optical density was determined using a microplate reader (Thermo max, Molecular Devices, CA). Results were calculated using the software provided with the microplate reader and expressed as pg/ml of cytokine produced in the supernatants. Results were expressed as a ratio of sample to control (S/C), where S is the amount of cytokine produced in PBMC stimulated with PHA plus test sample and C is the amount of cytokine produced in PBMC stimulated with PHA alone. S/C values were calculated for each cytokine measured. Some of the data are presented in Table 5, below, wherein each S/C value shown represents a single determination.

In vitro stimulation of spleen cell proliferation and cytokine production for the PGRG1 and PGRG1-5K is shown in Table 5 below.

TABLE 5
Spleen Cell Proliferation and Cytokine Production

| | | Spleen cell | Cytokine | Cytokine |
|---|---|---|---|---|
| | Concentration | proliferation | production | production |
| Sample | (ug/ml) | C3H/HeJ (S/C) | IL-6, S/C | G-CSF (S/C) |
| PGRG1 | 200 | 12.7 | 3.2 | 13.7 |
| | 150 | 11.1 | 3.3 | 9.7 |
| | 100 | 8.2 | 2.0 | 6.3 |
| | 30 | 4.4 | 1.9 | 2.8 |
| | 10 | 2.2 | 1.2 | 2.0 |
| | 3 | 1.7 | 1.4 | 1.9 |
| PGRG1 -5K | 200 | 15.8 | 7.8 | 43.6 |
| | 150 | 13.7 | 7.4 | 42.6 |
| | 100 | 11.1 | 5.8 | 35.3 |
| | 30 | 6.0 | 5.4 | 25.3 |
| | 10 | 4.5 | 4.4 | 16.7 |
| | 3 | 2.3 | 3.4 | 7.2 |

PGRG1 was observed to stimulate both mouse spleen cells and human peripheral blood monocytes to produce granulocyte colony stimulating factor (G-CSF) and other cytokines such as IL-6. Because G-CSF was also shown to accelerate the regeneration of renal tubular epithelial cells and prevent their apoptosis in a cisplatin-induced model of acute renal failure in mice, it is believed that PGRG1 induces an elevation of G-CSF which may be responsible, at least in part, for its renal protective effect.

### Example 5

Myelosuppression is a serious problem encountered by patients undergoing radiotherapy or chemotherapy for certain cancers or to prevent transplant rejection. This condition leads to leukopenia/neutropenia, thrombocytopenia, anemia, and frequently, secondary complications such as dotting disorders, bacterial, viral, or fungal infections, general fatigue, and malaise. PGRG1 was examined to determine if it enhances the recovery of different blood components from radiation-induced depletion of peripheral blood cells in a murine model.

### Animals

Ten- to twelve-week-old female BALB/c mice (19-21 g) were obtained from Charles River Laboratories (Wilmington, Mass.) and maintained on standard food pellets and water containing Neomycin (80 mg/L, Sigma Chemical Co., St. Louis, Mo.). Neomycin treatment was started for 5-7 days prior to the start of experiments.

### Controls

Granulocyte-Colony Stimulating Factor (G-CSF; Neupogen, Filgrastim) was obtained from Amgen (Thousand Oaks, Calif.) and diluted in sterile saline at 10 μg/ml immediately before use each day. It was used at 100 μg/kg/day (approximately 2 μg/mouse/day), a minimum effective dose described in a previous study (23). Sterile saline (0.9% Sodium Chloride Inj., USP, NDC 0074-4888-99) used for control animals and drug preparations was obtained from Abbott Laboratories (North Chicago, Ill.).

### Treatment Protocols

On day 0, groups of 5-6 mice each received 450 cGy of X-irradiation in a Philips 250 kvp X-Ray machine (Stanford University, Palo Alto, Calif.). The treatment was given in a split dosing regimen starting 4-5 hrs later on the same day (day 0) and then at approximately the same time each day thereafter on days 1-4, 7-9, 14-16, 21-23, and 28. No significant difference was observed whether animals were dosed according to this split regimen or daily for 28 days (comparative data in Experiment/Computer File HEM4 and Notebook #160). Control groups included (a) normal, unirradiated, untreated mice (baseline control for weight comparison, data not shown), (b) irradiated mice that received subcutaneous injections of saline (negative control), or (c) irradiated mice that received subcutaneous injections of 100 μg/kg/day of G-CSF (positive control), demonstrating that G-CSF was effective (data not shown).

All animals were weighed before each injection, and 20 to 50 μl of blood was obtained from the tail vein twice a week for 4 weeks starting on day 4 post-irradiation. Animals maintained their normal weights (versus normal, unirradiated, untreated mice) throughout the experimental period.

### Peripheral Blood Counts

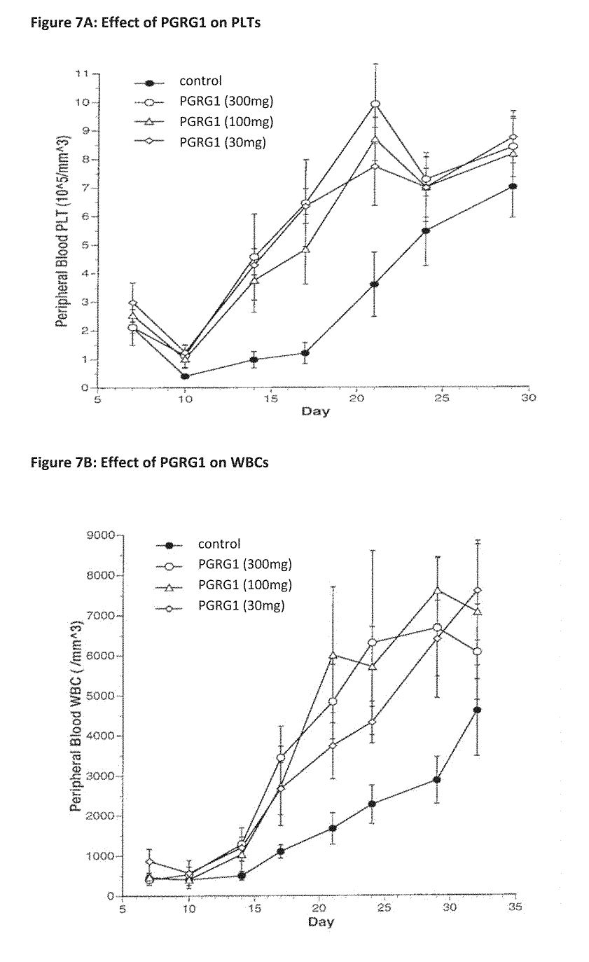

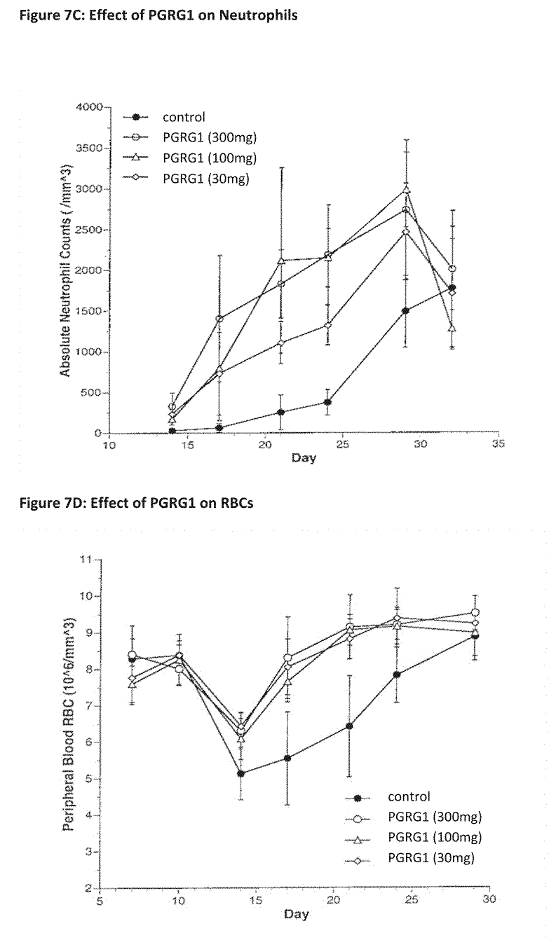

Peripheral blood parameters including platelets (PLT), white blood cells (WBC) and red blood cells (RBC) were measured using a Serono System 9000 Automated Cell Counter (Baker Diagnostics, Allentown, Pa.). Blood smears were made on glass slides, stained with the Leukostat Stain Kit (No. CS-430, Fisher Scientific, Wilmington, Pa.), and differential WBC counts were done under a Nikon Labophot-2 microscope. Statistical analyses were done using the unpaired t test or Mann-Whitney nonparametric test. The results for the effect of PGRG1 on platelets (PLT), white blood cells (WBC), neutrophils and red blood cells (RBC) are summarized in FIGS. 7A-7D.

Clinical Trials of Cancer Patients Using PGRG1

| Patient | Age | Cancer | Cancer Stage | diagnosed Time |
|---|---|---|---|---|
| Patient 1 | Male, 63 | Lung Cancer adenocarcinoma | IV | May 1, 2012 |
| Patient 2 | Male 56 | Small Cell Lung Cancer | IIIB | May 8, 2012 |

Patient 1 Results

| | Use of | WBC | RBC | PIT |
|---|---|---|---|---|
| Chemotherapy | PGRG1 | (Normal 4-10) | (Normal 3.5-5.5) | (Normal 100-300) |
| Before | None | 7.87 | 4.56 | 264 |
| After 1^{st} & 2^{nd} | None | 3.88 | 3.0 | 212 |
| After 3rd | Used | 9.8 | 3.21 | 237 |
| After 4th | Used | 7.0 | 4.84 | 582 |
| After 5th | Used | 6.78 | 3.92 | 279 |
| After 6th | Used | 10.0 | 4.42 | 277 |

Patient 2 Results

| | Use of | WBC | RBC | PIT |
|---|---|---|---|---|
| Chemotherapy | RG-1 | (Normal 4-10) | (Normal 3.5-5.5) | (Normal 100-300) |
| Before | None | 8.9 | 4.75 | 300 |
| After 1^{st} | None | 2.6 | 3.3 | 125 |
| After 2nd | Used | 6.0 | 3.85 | 170 |
| After 3rd | Used | 3.8 | 4.52 | 217 |
| After 4th | Used | 3.8 | 2.99 | 148 |
| After 5th | Used | 4.8 | 3.63 | 228 |
| After 6^{th} | Used | No test | No test | No test |

Patient 1 took 10 capsules of PGRG1 (450 mg of PGRG1 per capsule) every day, 5 o'clock in the morning and 5 o'clock in the evening, after his 2^{nd} chemotherapy. After the 1^{st} and 2^{nd} chemotherapy and before taking PGRG1, patient 1 felt weak, had no appetite, and reported his stomach was uncomfortable. After taking PGRG1, patient 1 reported that weakness, loss of appetite and stomach discomfort returned to normal by the 6^{th} week of treatment.

Patient 2 took 8 capsules of PGRG1 (450 mg of PGRG1 per capsule) every day, 4 o'clock in the morning and 4 o'clock in the evening, after his 1st chemotherapy. After the 1^{st} chemotherapy and before taking PGRG-1, patient 2 reported that he felt weak, sweaty, and had no appetite. After taking PGRG-1, the patient indicated that weakness, loss of appetite and body sweats had returned to normal by the 6^{th} week.

While various specific embodiments have been illustrated and described, it will be appreciated that equivalents of the specifically disclosed materials and techniques will also be applicable and various changes can be made without departing from the spirit and scope of the invention(s); such equivalents are intended to be included within the following claims. Therefore, it is to be understood that the disclosure is not to be limited to the specific embodiments disclosed herein, as such are presented by way of example. Although specific terms are employed herein, they are used in a generic and descriptive sense only and not for purposes of limitation.

All literature and similar materials cited in this application, including, but not limited to, patents, patent applications, articles, books, treatises, internet web pages and other publications cited in the present disclosure, regardless of the format of such literature and similar materials, are expressly incorporated by reference in their entirety for any purpose to the same extent as if each were individually indicated to be incorporated by reference. In the event that one or more of the incorporated literature and similar materials differs from or contradicts the present disclosure, including, but not limited to defined terms, term usage, described techniques, or the like, the present disclosure controls.

What is claimed is:

1. An isolated polygalacturonan rhamnogalacturonan 1 (PGRG1) composition obtained from the roots of Astragalus membranaceus, wherein the PGRG1 in the composition has a weight average molecular weight (waMW) of 10 kDa to 50 kDa and a rhamnose-to-galacturonic acid (Rha:GalA) mole percent ratio of at least 0.06.

2. The isolated PGRG1 composition of claim 1 wherein the PGRG1 in the composition has a backbone structure of [1,4 and 1,2-linked RhaGalA]ₘ interspersed with [1,4-linked GalAGalA]ₙ.

3. The isolated PGRG1 composition of claim 1 where the Astragalus membranaceus variety is selected from Astragalus membranaceus (Fisch.) Bge. and Astragalus membranaceus (Fisch.) var. mongholicus (Bge.) Hsiao species.

4. The isolated PGRG1 composition of any of claims 1 to 3 wherein the Astragalus membranaceus was grown in a province of Peoples' Republic of China selected from Shanxi, Inner Mongholia, Gansu, Hebei and Liaoning.

5. The isolated PGRG1 composition of any of claims 1 to 4 where the roots are from cultivated Astragalus membranaceus plants between two and three years old.

6. The isolated PGRG1 composition of any of claims 1 to 4 where the roots are from wild-type Astragalus membranaceus plants between three and ten years old.

7. The isolated PGRG1 composition of any of claims 1 to 4 where the roots are from cultivated Astragalus membranaceus plants up to two years old.

8. A PGRG1-5K composition, having a weight average molecular weight range of 40 to 120 kDa, wherein the PGRG1-5K composition is purified from the PGRG1 composition of any of claims 1 to 7.

9. An oral PGRG1 formulation comprising:
(a) a therapeutically effective amount of the isolated PGRG1 composition of any one of claims 1 to 7 or the PGRG1-5K composition of claim 8; and, optionally,
(b) a pharmaceutically suitable excipient.

10. A method of producing the purified PGRG1 composition of claim 1, comprising:
(a) extracting the dried roots of Astragalus membranaceus in an aqueous solution at about 100° C. over a total of nine hours; and
(b) adding to the extract from step (a) sufficient lower alkanol to precipitate the PGRG1 composition, and isolating the precipitated PGRG1 composition.

11. A method of producing the isolated PGRG1-5K composition of claim 8, comprising:
(a) subjecting an aqueous solution of the isolated PGRG1 composition of any one of claims 1 to 7 to ultrafiltration through a filter having a 5 kDa molecular weight cutoff; and
(b) isolating the PGRG1-5K composition from the retentate from step (a).

12. A method of treating kidney disease in a mammal by reducing blood creatinine levels, comprising orally administering to the mammal an amount of the isolated PGRG1 composition of any of claims 1 to 7, the PGRG1-5K composition of claim 6, or the oral PGRG1 formulation of claim 8 effective to reduce blood creatinine levels as compared to blood creatinine levels in an untreated mammal.

13. A method of inducing hematopoiesis in a mammal, as measured by increased blood cell counts, by orally administering to the mammal an amount of the purified PGRG1 composition of any of claims 1 to 7, the PGRG1-5K composition of claim 6, or the oral PGRG1 formulation of claim 8 effective to induce hematopoiesis.

14. A method of treating immune system dysfunction by orally administering to a mammal an amount of the purified PGRG1 composition of any of claims 1 to 7, the PGRG1-5K composition of claim 6, or the oral PGRG1 formulation of claim 8 effective to treat immune system dysfunction.

15. The method of claims 12-14 where the mammal is a human.

16. The method of claim 12, further comprising administering at least one additional therapeutic agent capable of treating or ameliorating kidney disease.

17. The method of claim 13, further comprising administering at least one additional therapeutic agent capable of inducing hematopoiesis.

18. The method of claim 14, further comprising administering at least one additional therapeutic agent capable of improving immune function.

19. A dietary supplement comprising the purified PGRG1 composition of any of claims 1 to 7, or the PGRG1-5K composition of claim 8.
